(19) United States
(12) Patent Application Publication
(10) Pub. No.: US 2015/0105267 A1
(43) Pub. Date: Apr. 16, 2015

(54) WHOLE GENOME SEQUENCING OF A HUMAN FETUS

(71) Applicant: UNIVERSITY OF WASHINGTON THROUGH ITS CENTER FOR COMMERCIALIZATION, Seattle, WA (US)

(72) Inventors: Jay Ashok Shendure, Seattle, WA (US); Jacob Otto Kitzman, Seattle, WA (US); Matthew Snyder, Seattle, WA (US)

(21) Appl. No.: 14/403,558

(22) PCT Filed: May 24, 2013
(86) PCT No.: PCT/US2013/042774

§ 371 Date: Nov. 24, 2014

Related U.S. Application Data
(60) Provisional application No. 61/651,356, filed on May 24, 2012.

(51) Int. Cl. C12Q 1/68

(52) U.S. Cl. CPC C12Q 1/6874
USPC 506/2

(57) ABSTRACT

Methods of genome sequencing of a fetus are provided herein. In some embodiments, such methods include steps of predicting inheritance or transmission of an allele from one or more maternal-only heterozygous sites from a maternal genomic sequence to a fetal genome sequence; and predicting inheritance or transmission of an allele from one or more paternal-only heterozygous sites from a paternal genomic sequence to a fetal genome sequence. In some embodiments, the methods may also include predicting transmission of one or more genomic variants at one or more heterozygous sites that are present on both a maternal genomic sequence and a paternal genomic sequence. According to these embodiments, the paternal genomic sequence and the maternal genomic sequence are derived from a biological sample containing DNA. According to other embodiments, the sequencing methods may include a step of predicting de novo mutations in a fetal genomic sequence.

### PRIORITY CLAIM

This application claims priority to U.S. Provisional Application No. 61/651,356, filed May 24, 2012, the subject matter of which is hereby incorporated by reference as if fully set forth herein.

### STATEMENT OF GOVERNMENT INTEREST

The present invention was made with government support under Grant No. HG006283 awarded by the National Institutes of Health/National Human Genome Research Institute. The Government has certain rights in the invention.

### BACKGROUND

On average, ˜13% of cell-free DNA isolated from maternal plasma during pregnancy is fetal in origin (Nygren et al. 2010). The concentration of cell-free fetal DNA in maternal circulation varies between individuals, increases across gestation, and is rapidly cleared postpartum (Lo et al. 1998; Lo et al. 1999). Despite this variability, cell-free fetal DNA has been successfully targeted as a resource for non-invasive prenatal diagnosis, including the development of targeted assays for single gene disorders (Lo & Chiu 2007). More recently, several groups demonstrated shotgun, massively parallel sequencing of cell-free DNA from maternal plasma as a robust approach for non-invasively diagnosing fetal aneuploidies such as trisomy 21 (Fan et al. 2008; Chiu et al. 2008).

Although a prenatal test to non-invasively sequence a whole fetal genome would improve the state of the art dramatically, several technical remain for this goal to be achieved using cell-free DNA from maternal plasma. First, the sparse representation of fetal-derived sequences poses the challenge of detecting low frequency alleles inherited from the paternal genome as well as those arising from de novo mutations in the fetal genome (from an analytical perspective, analogous to the challenge of detecting subclonal somatic mutations in a tumor). Additionally, maternal DNA predominates in plasma, obscuring the ability to assess maternally inherited variation at individual sites in the fetal genome.

Therefore, it would be desirable to produce methods that would make it possible to non-invasively predict the whole genome sequence of a fetus to high accuracy and completeness, thereby facilitating the comprehensive prenatal diagnosis of Mendelian disorders and obviating the need for invasive prenatal diagnostic procedures with their attendant risks.

### SUMMARY

Methods of genome sequencing of a fetus are provided herein. In some embodiments, such methods include steps of predicting inheritance or transmission of an allele from one or more maternal-only heterozygous sites from a maternal genomic sequence to a fetal genome sequence; and predicting inheritance or transmission of an allele from one or more paternal-only heterozygous sites from a paternal genomic sequence to a fetal genome sequence. In some embodiments, the methods may also include predicting transmission of one or more genomic variants at one or more heterozygous sites that are present on both a maternal genomic sequence and a paternal genomic sequence. According to these embodiments, the paternal genomic sequence and the maternal genomic sequence are derived from a biological sample containing DNA.

In one embodiment, predicting inheritance or transmission of an allele from one or more maternal-only heterozygous sites from a maternal genomic sequence includes one or more steps of sequencing a maternal genomic sequence derived from a maternal biological sample; sequencing a plurality of maternal-fetal cell-free plasma DNA sequences derived from a maternal plasma sample obtained during pregnancy; determining a percentage of fetal DNA in the maternal plasma sample; phasing the one or more maternal-only heterozygous sites present in the maternal genomic sequence into one or more haplotype blocks, and predicting inheritance or transmission of one or more haplotype blocks using a maternal Hidden Markov Model (HMM).

In another embodiment, predicting inheritance or transmission of an allele from one or more paternal-only heterozygous sites from a paternal genomic sequence includes one or more steps of sequencing a paternal genomic sequence derived from a paternal biological sample; sequencing a plurality of maternal-fetal cell-free plasma DNA sequences derived from a maternal plasma DNA sample obtained during pregnancy; determining a percentage of fetal DNA in the maternal plasma DNA sample; phasing the one or more paternal-only heterozygous sites present in the maternal genomic sequence into one or more haplotype blocks, and predicting inheritance or transmission of one or more haplotype blocks using a paternal HMM.

According to some embodiments, the sequencing methods may also include a step of predicting de novo mutations in a fetal genomic sequence. This method may include one or more steps of sequencing a paternal genomic sequence from a paternal biological sample; sequencing a maternal genomic sequence from a maternal biological sample; sequencing a plurality of maternal-fetal DNA sequences from a maternal plasma sample; comparing the paternal genomic sequence and the maternal genomic sequence to the maternal-fetal DNA sequences; identifying one or more candidate de novo alleles as variant alleles observed in the maternal-fetal DNA sequences but are not observed in the maternal genomic sequence or the paternal genomic sequence; and applying a set of filters to the one or more candidate de novo alleles to remove known variants and artifacts from sequencing or mapping, wherein one or more remaining candidate de novo alleles comprise at least one true de novo mutation.

### BRIEF DESCRIPTION OF THE DRAWINGS

FIG. 1 illustrates an experimental approach for predicting a fetal genome sequence according to some embodiments. (A) is a schematic of sequenced individuals in a family trio. Maternal plasma sequences were ˜13% fetal-derived based on read depth at chrY and alleles specific to each parent. (B) shows inheritance of maternally heterozygous alleles inferred using long haplotype blocks. Among plasma sequences, maternal-specific alleles are more abundant when transmitted (expected 50% versus 43.5%), but there is significant overlap between the distributions of allele frequencies when taking considering sites in isolation (left histogram, yellow=shared allele transmitted, green=maternal-specific allele transmitted). Taking average allele balances across haplotype blocks (right histogram) provides much greater separation, permitting more accurate inference of maternally transmitted alleles. (C) is a histogram of fractional read depth among plasma data at paternal-specific heterozygous sites. In the overwhelming majority of cases when the allele specific to the father was not detected, the opposite allele had been transmitted (96.8%, n=561,552). (D) shows that de novo missense mutation in the gene ACMSD detected in 3 of 93 maternal plasma reads and later validated by PCR and resequencing. The mutation, which is not observed in NCBI's public database of SNPs (“dbSNP,” see http://www.ncbi.nlm.nih.gov/projects/SNP/) nor among >4,000 individuals' coding exons sequenced as part of the NHLBI Exome Sequencing Project (http://evs.gs.washington.edu), creates a leucine-to-proline substitution at a site conserved across all aligned mammalian genomes (UCSC Genome Browser) in a gene implicated in Parkinson's disease by genome-wide association studies (Nalls et al. 2011).

FIG. 2 illustrates coverage of autosomes and sex chromsomes and estimation of percent fetal contribution to maternal plasma sequences according to some embodiments. For each individual sequenced in two trios, relative read depth (log-scaled) is shown, taking only reads at 30-mer positions unique within the genome (Lo et al. 1999) and dividing by the overall autosomal mean to scale autosomes to an average of 100%. Chromsomes X and Y are expected to be at 50% each in males, or, 100% and 0%, respectively, in females. Normalized abundances of chrY are highlighted are indicated in blue; because each fetus was male, scaling these relative percentage of chrY reads from the plasma sequencing by a factor of two yields an estimate for the abundance of fetal DNA.

FIG. 3 illustrates the accuracy of fetal genotype inference from maternal plasma sequencing according to some embodiments. Accuracy is shown for paternal-only heterozygous sites, and for phased maternal-only heterozygous sites, either using maternal phase information (black) or instead predicting inheritance on a site-by-site basis (gray).

FIG. 4 illustrates that HMM-based predictions correctly predict maternally transmitted alleles across ˜1 Mbp on chromosome 10 according to some embodiments, despite site-to-site variability of allelic representation among maternal plasma sequences (dots, right axis).

FIG. 5 shows an example of HMM-based detection of recombination events or haplotype assembly switch errors according to some embodiments. A maternal haplotype block of 917 Kbp on chromosome 12q is shown, with dots/points representing the frequency of haplotype A alleles among plasma reads, and the black line indicating the posterior probability of transmission for haplotype A computed by the HMM at each site. A block-wide odds ratio test (OR) predicts transmission of the entire haplotype B, resulting in incorrect prediction at 272 of 587 sites (46.3%). The HMM predicts a switch between chromosomal coordinates 115,955,900 and 115,978,082, and predicts transmission of haplotype B alleles from the centromeric end of the block to the switch point, and haplotype A alleles thereafter, resulting in correct predictions at all 587 sites. All three overlapping informative clones support the given maternal phasing of the SNPs adjacent to the switch site (not shown), suggesting that the switch predicted by the HMM results from a maternal recombination event rather than an error of haplotype assembly.

FIG. 6 illustrates the accuracy of maternal transmission inference as a function of haplotype block size according to some embodiments. Maternal-only heterozygous sites were ranked in decreasing order by haplotype block size (the number of other maternal-heterozygous sites phased in the same block). Blue dotted lines denote cutoffs retaining 95% of sites. (A) shows cumulative distribution of maternal-only heterozygous sites by block; 95% of such sites are contained in the top 45% of haplotype blocks. (B) and (C) show cumulative accuracy among maternal-only heterozygous sites ranked by block size. Cumulative accuracy is 99.7% among the 95% of sites in the largest haplotype blocks, and falls to 99.3% when the remaining 5% of sites are included. (B) shows cumulative accuracy including all blocks. (C) shows cumulative accuracy when removing the largest block, which resides among a duplication-rich region at 43.7 Mb-44.3 Mb on chromosome 17q.

FIG. 7 shows simulation of effects of reduced coverage, haplotype length, and fetal DNA concentration on fetal genotype inference accuracy according to some embodiments, wherein fetal genotype inference accuracy is defined as the percentage of sites at which the inherited allele was correctly identified out of all sites where prediction was attempted. Heat maps of accuracy after in silico fragmentation of haplotype blocks and (A) shallower sequencing of maternal plasma, or (B) reduced fetal concentration among plasma sequences.

FIG. 8 shows inference accuracy for paternal transmission at paternal-only heterozygous sites, as a function of plasma shotgun sequencing depth according to some embodiments (median=78-fold). Inference accuracy is generally higher at more deeply sequenced sites. At sites with low overall coverage, too few fetal-derived reads are sampled, and the paternally transmitted allele is more likely to go unobserved. On the other end, sites with extremely high coverage may reside in regions of high copy number or dense repeat content that are recalcitrant to accurate mapping and variation calling with short reads.

FIG. 9 illustrates that shared heterozygous sites are primarily common polymorphisms according to some embodiments. Population minor allele frequency (MAF) from the 1000 Genomes Project (Lo et al. 1998) was determined for each heterozygous site in trio “I1”. Sites were categorized as present only in I1-P (“Paternal-only”, blue), only in I1-M (“Maternal-only”, green), or in both (“Shared”, purple), and their counts shown as a function of MAF. As expected, rare heterozygous sites (MAF less than 5%, n=3.42×10⁵) are overwhelmingly found in one parent or the other (92.8%) but not both. Moreover, shared sites are as a whole significantly more common in the population than either parent-specific subset (P<2.2×10₋₁₆, Mann-Whitney rank sum test).

FIG. 10 illustrates results for detecting sites of de novo mutation among maternal fetal plasma sequences according to some embodiments. Shown on a log 10 scale are counts of candidate and true single-base de novo substitution variants remaining after application of successive quality filters (filled gray area and dots, respectively).

FIG. 11 is an overview of noninvasive fetal whole genome sequencing according to some embodiments. (A) shows sample collection. Parental blood samples are collected in the first or second trimester. After centrifugation, parental DNA is extracted from peripheral blood mononuclear cells (PBMC) or buffy coat, while cfDNA is isolated from the maternal plasma. (B) shows sample processing. Extracted DNA is amplified for library preparation and sequenced to high depth. Reads are aligned to a reference genome to identify variant alleles carried by one or both parents. (C) shows inference of fetal genome. A statistical model combines known parental genotypes and alleles observed in cfDNA reads to predict fetal inheritance. High-impact mutations, whether inherited or de novo, are identified (lollypop). (D) shows Interpretation. Identified variants are compared with catalogs of known disease-associated mutations. (E) shows confirmation. A subset of clinically actionable predicted mutations is confirmed with conventional procedures such as amniocentesis. Accuracy of genome inference can be assessed post hoc with DNA extracted from cord blood after delivery

FIG. 12 is a schematic illustrating inference of the fetal genome from haplotype blocks according to one embodiment. (a) Phasing of maternal heterozygous sites into haplotype blocks (red bars). Haplotype blocks contain dozens or hundreds of such sites and cover over 300 kilobases on average. A single chromosome may have over 100 haplotype blocks; contiguity between blocks is not defined. Approximately 90% of all heterozygous sites are incorporated into haplotype blocks. Sites shown do not represent real data. (b) Phasing of paternal heterozygous sites into haplotype blocks (blue bars). Paternal and maternal blocks may overlap but are independently defined. (c) Schematic of inference of fetal inheritance of maternal haplotype blocks. Numbers shown assume a constant sequencing depth of 100× at each. After sequencing the cfDNA, evidence of deviations from expected allele counts is aggregated over each site in haplotype blocks ‘A’ and ‘B’, and the more likely block is predicted. Block-level predictions in turn determine predictions at each contained site. The site in the center of the block would be incorrectly predicted if sites were considered independently; its inclusion in a haplotype block mitigates sampling noise and corrects the prediction. (d) Schematic of inference of fetal inheritance of paternal haplotype blocks. Numbers are presented as in (c). The observed ‘G’ allele at the rightmost site, likely to cause an incorrect prediction if sites were considered independently, is now correctly identified as an error introduced during the sequencing process rather than evidence of transmission of the ‘G’ allele. (e) The inferred fetal genome is a composite of the parental haplotype blocks

### DETAILED DESCRIPTION

Methods for a comprehensive prediction and detection of a fetal genome sequence using cell-free fetal-derived DNA from maternal plasma are provided herein. Such methods may include predicting inheritance of one or more parental genetic abnormalities (i.e., maternal and/or paternal abnormalities), predicting or detecting the presence of one or more de novo mutations in a fetal genome, or both. In some aspects, the methods are based on genomic sequences that are non-invasively obtained from a subject. In some embodiments, the methods described herein may be carried out by a computer or computer system.

According to some embodiments, methods for prediction and detection of a fetal genome sequence include a method of predicting inheritance of one or more parental genetic abnormalities (i.e., maternal and/or paternal abnormalities). In one aspect of this embodiment, the methods are directed to prediction of inheritance of one or more single nucleotide variants (“SNPs”), which are the most common form of both non-pathogenic and pathogenic genetic variation in human genomes (Durbin 2010; Stenson et al. 2009). Other inheritable genetic abnormalities that may be suitable for predictions using the methods described herein include, but are not limited to, a missense mutation, a nonsense mutation, a deletion, an insertion, copy number change, a frame shift mutation or other abnormalities.

The methods for predicting inheritance of parental genetic abnormalities may include one or more of the following steps: predicting “maternal-only” inheritance of one or more genetic abnormalities (i.e., abnormalities found in the maternal genome but not the paternal genome), predicting “paternal-only” inheritance of one or more genetic abnormalities (i.e., abnormalities found in the paternal genome but not the maternal genome), and predicting dual parental inheritance of one or more genetic abnormalities (i.e., abnormalities found in both the paternal genome and the maternal genome).

According to certain aspects of the methods for predicting inheritance of parental genetic abnormalities, maternal-only inheritance of one or more genetic abnormalities (e.g., single nucleotide polymorphisms, or SNPs) may include, among other things, predicting inheritance or transmission of an allele (variant or non-variant) from one or more maternal-only heterozygous sites from a maternal genomic sequence to the fetal genome sequence.

Predicting inheritance or transmission of a variant allele from one or more maternal-only heterozygous sites may be accomplished by one or more steps including, but not limited to, sequencing a maternal genomic sequence from a maternal genomic DNA sample (e.g., cellular genomic DNA in a blood sample or other maternal biological sample), sequencing one or more maternal-fetal cell-free plasma DNA sequences from a maternal DNA sample (i.e. from a maternal blood or plasma sample), determining a percentage of fetal DNA in a maternal DNA sample obtained from a female subject, assembling or phasing the maternal-only heterozygous sites or variant alleles (e.g., SNPs) present in the paternal genomic sequence into one or more haplotype blocks (or “phased blocks”), and predicting inheritance or transmission of one or more haplotype blocks using a Hidden Markov Model (HMM). In some aspects, the HMM can also be used to infer transitions within the one or more haplotype blocks. Inferred transitions represent either true recombination events or switch errors in maternal phasing, and can therefore be used in methods predict and map sites of such recombination events or switch errors within a maternal haplotype.

According to some embodiments, an HMM that can be used to predict inheritance or transmission of one or more haplotype blocks or to infer transitions within phased blocks of a maternal genome sequence (i.e., a maternal HMM) is provided. In some embodiments, the maternal HMM includes, but is not limited to, a set of one or more latent inheritance states (i.e., unobserved states) at each maternal heterozygous site, a probability model for transitions between latent states, and an emission probability model for each latent state.

A latent inheritance state defines which of two sites or haplotype blocks is inherited at each site. According to some embodiments, an emission probability model may be designed for each latent state. In one such embodiment, an emission probability model may include one or more steps of calculating a probability of observing a first maternal-inherited allele that is the same as a paternal-inherited homozygous allele from a maternal heterozygous site; and calculating a probability of observing a second maternal-inherited allele that differs from a paternal-inherited homozygous allele from a maternal heterozygous site. Some aspects of calculating such probabilities are described in detail in Example 1, and may include a calculation of a probability (Pr) of observing said first and second maternal-inherited alleles (k) among N total reads with a fetal percentage F using Equation 1 and Equation 2 (below), respectively.

$$\begin{matrix} Pr ( K = k \vert N , F ) = Bin ( N , \frac{1 - F}{2} + \frac{F}{2} + \frac{F}{2} ) & ( Equation 1 ) \\ Pr ( K = k \vert N , F ) = Bin ( N , \frac{1 - F}{2} + \frac{F}{2} ) = Bin ( N , 0.5 ) & ( Equation 2 )\end{matrix}$$

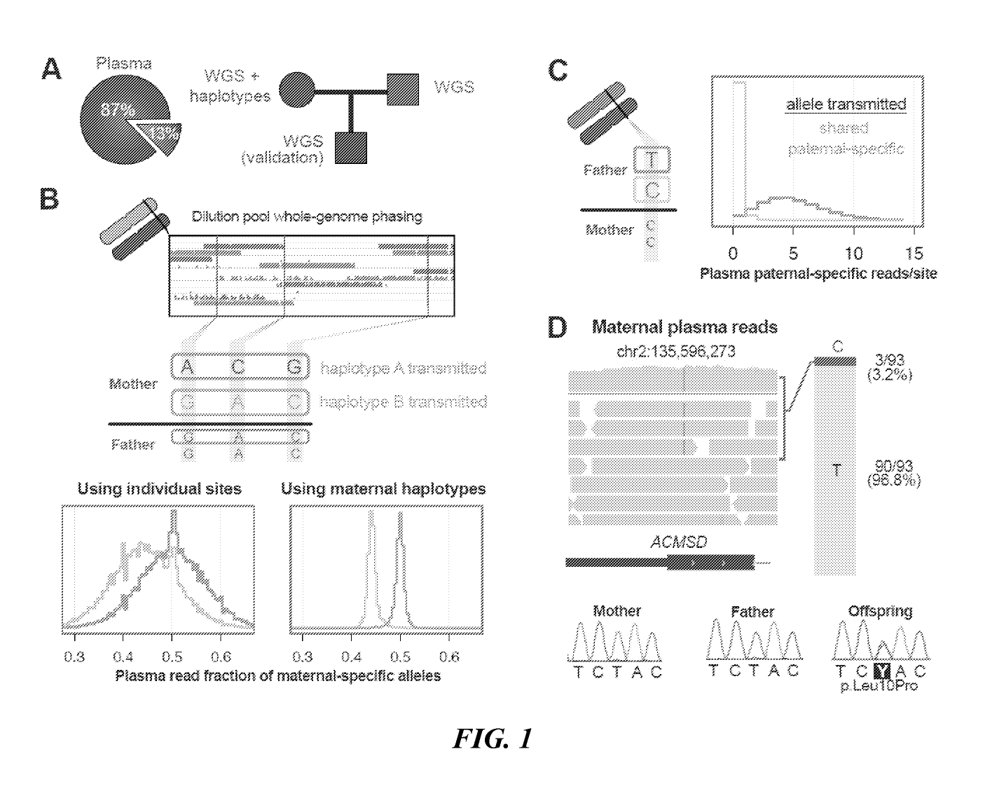

According to other aspects of the methods for predicting inheritance of parental genetic abnormalities, paternal-only inheritance of one or more genetic abnormalities (e.g., single nucleotide polymorphisms, or SNPs) may include, among other things, predicting inheritance or transmission of an allele (variant or non-variant) from one or more paternal-only heterozygous sites from a paternal genomic sequence to the fetal genome sequence. As described in the Examples below, the observation (or lack thereof) of paternal alleles in shotgun libraries derived from maternal plasma was used to predict paternal transmission (FIG. 1C).

Predicting inheritance or transmission of a variant allele from one or more paternal-only heterozygous sites may be accomplished by one or more steps including, but not limited to, sequencing a paternal genomic sequence from a paternal genomic DNA sample (e.g., cellular genomic DNA in a blood sample or other paternal biological sample), sequencing a maternal genomic sequence and one or more maternal-fetal cell-free plasma DNA sequences from a maternal DNA sample derived from a maternal blood sample, determining a percentage of fetal DNA in the maternal DNA sample, assembling or phasing paternal-only heterozygous sites or variant alleles (e.g., SNPs) present in the paternal genomic sequence into one or more haplotype blocks (or “phased blocks”), and predicting inheritance or transmission of one or more haplotype blocks using a Hidden Markov Model (HMM). In some aspects, the HMM can also be used to infer transitions within the one or more haplotype blocks. Inferred transitions represent either true recombination events or switch errors in paternal phasing, and can therefore be used in methods predict and map sites of such recombination events or switch errors within a paternal haplotype.

According to some embodiments, an HMM that can be used to predict inheritance or transmission of one or more haplotype blocks or to infer transitions within phased blocks of a paternal genome sequence (i.e., a paternal HMM) is provided. In some embodiments, the paternal HMM includes, but is not limited to, a set of one or more latent inheritance states (i.e., unobserved states) at each maternal heterozygous site, a probability model for transitions between latent states, and an emission probability model for each latent state.

A latent inheritance state defines which of two sites or haplotype blocks is inherited at each site. According to some embodiments, an emission probability model may be designed for each latent state. In one such embodiment, an emission probability model may include one or more steps of calculating a probability of observing a first paternal-inherited allele that is the same as a maternal-inherited homozygous allele from a paternal heterozygous site; and calculating a probability of observing a second paternal-inherited allele that differs from a maternal-inherited homozygous allele from a paternal heterozygous site. Some aspects of calculating such probabilities are described in detail in Example 1, and may include a calculation of a probability (Pr) of observing said first and second paternal-inherited alleles (k) among N total reads with a fetal percentage F using Equation 3 and Equation 4 (below), respectively (c is a small number representing the probability of a sequencing or technical error).

$$\begin{matrix}Pr ( K - k \vert N , {F}^{′} ) - Bin ( N , 1 - g ) & ( Equation 3 ) \\ Pr ( K - k \vert N , F ) - Bin ( N , \frac{F}{2} ) & ( Equation 4 )\end{matrix}$$

According to other aspects of the methods for predicting inheritance of parental genetic abnormalities, dual parental inheritance of one or more genetic abnormalities (e.g., single nucleotide polymorphisms, or SNPs) may include, among other things, predicting inheritance or transmission of an allele (variant or non-variant) from one or more heterozygous sites found in both a maternal and a paternal genome sequence to the fetal genome sequence as described in detail in the Examples below. Additionally, fetal genotypes are trivially predicted at sites where the parents are both homozygous (for the same or different allele).

In some embodiments, inheritance of the maternal genomic sequence, the paternal genomic sequence, or both are analyzed using a haplotype-resolved genome sequencing method. According to such embodiments, a maternal genomic sequence, a paternal genomic sequence, or both, are haplotype-resolved genome sequences derived from a blood or plasma sample from the mother (i.e., a maternal blood or plasma sequence), the father (i.e., a paternal blood or plasma sequence) or from both parents, respectively. A haplotype-resolved genome sequence is a map of haplotypes, which are represented by one or more inherited clusters, or “blocks” of SNPs. As described in Example 1 below, allelic imbalance in maternal plasma which manifested across experimentally determined maternal haplotype blocks was used to predict said blocks' maternal transmission (FIG. 1B). Similarly, paternal haplotype blocks were experimentally determined to predict paternal transmission (Example 3).

The haplotype-resolved genome sequence may be determined by assembling (or “phasing,” “molecular phasing”) variants or SNPs into one or more haplotype blocks by a suitable method or algorithm known in the art including, but not limited to a HapCUT algorithm (Bansal & Bafna 2009), a MixSIH model (see, e.g., Matsumoto & Kiryu), a family-based inference or pedigree (see, e.g., Roach et al. 2010), an other algorithms such as Greedy heuristic algorithm (Levy et al. 2007), HASH or Markov chain Monte Carlo (MCMC) algorithm (Bansal et al. 2008), a ReFHap algorithm (Duitama et al. 2010) or other known or experimental algorithms tailored to a particular set of data (see, e.g., He et al. 2010; Xie et al. 2012). Additionally, methods for experimentally determining haplotypes for both rare and common variation at a genome-wide scale have recently been demonstrated (Kitzman et al. 2011; Suk et al. 2011; Fan et al. 2011; Ma et al. 2010).

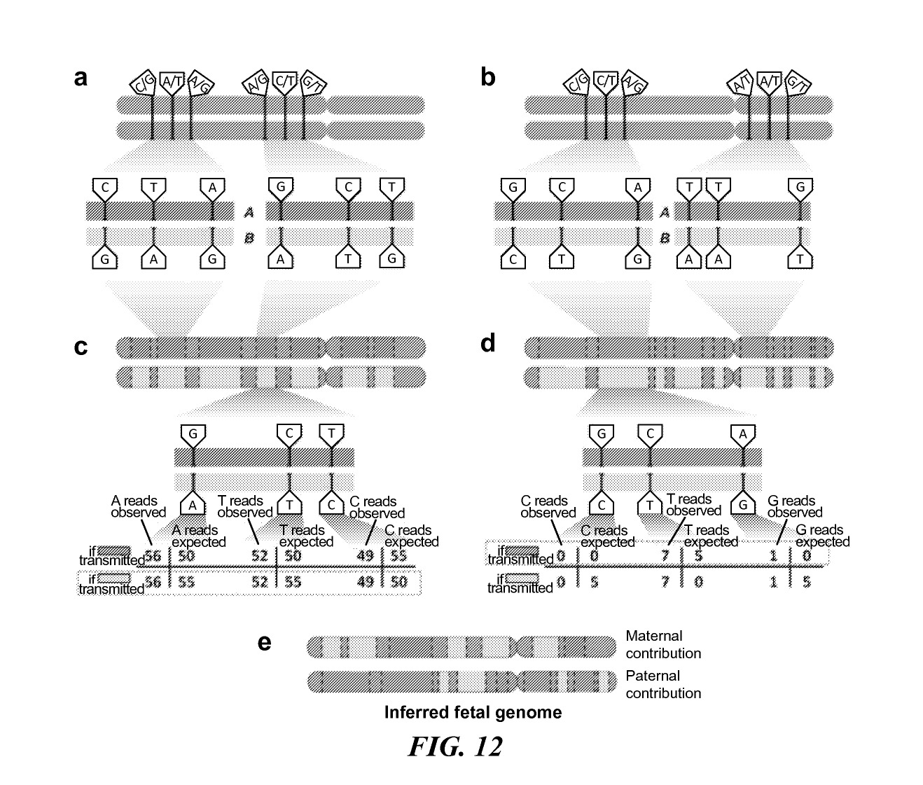

In certain embodiments, assembling or phasing the maternal-only heterozygous sites or variant alleles (e.g., SNPs) present in the paternal genomic sequence into one or more haplotype blocks (or “phased blocks”) is accomplished by a technique described below. Smaller subsections of haplotypes, or ‘haplotype blocks’ may be ascertained, wherein each haplotype block contains dozens or hundreds of heterozygous sites and covers tens to hundreds of kilobases. At a given locus, two haplotype blocks are defined, arbitrarily labeled ‘A’ and ‘B’, representing the grouping, or ‘phase’, of genetic variants present on the two homologs (FIG. 12a, 12b). Applying this technique to the parental genomes allows for evidence of transmission of whole blocks ‘A’ or ‘B’, instead of individual alleles ‘A’ or ‘B’, to be searched for by aggregating evidence of overrepresentation of each phased allele along the length of a haplotype block (FIG. 12c, 12d). The signal generated by jointly considering large blocks of sites helps to mitigate site-by-site noise that can occur. Moreover, sites at which both parents are heterozygous, where inheritance is particularly difficult to individually predict owing to the addition of a third possible fetal genotype, benefit from their inclusion in haplotype blocks with stronger evidence of inheritance. The inferred fetal genome, then, includes of a set of predictions about inheritance of one or the other haplotype block from each of the parental genomes (FIG. 12e).

Although it has been suggested that parental haplotypes may be exploited to detect allelic imbalance in maternal plasma across long segments of the genome to deduce blocks of inheritance in the fetal genome (Lo et al. 2010), this study was limited in at least the following ways. First, no technology existed to measure parental haplotypes experimentally at a genome-wide scale, thus the proposed method depended on the availability of parental haplotypes. Consequently, an invasive procedure, chorionic villus sampling (CVS), was used to obtain placental material for fetal genotyping. Second, parental genotypes and invasively obtained fetal genotypes were used to infer parental haplotypes, which were then used in combination with the sequencing of DNA from maternal plasma to predict the fetal genotypes. The circularity of these inferences makes it difficult to assess how well the method would perform in practice. Third, the analysis was restricted to several hundred thousand parentally heterozygous sites of common single nucleotide polymorphism (SNPs) represented on a commercial genotyping array. These common SNPs are only a small fraction of the several million heterozygous sites present in each parental genome, and include few of the rare variants that predominantly underlie Mendelian disorders (MacArthur et al. 2012). Fourth, no effort was made to ascertain de novo mutations in the fetal genome. As de novo mutations underlie a substantial fraction of dominant genetic disorders, their detection is important for comprehensive prenatal genetic diagnostics. Therefore, although this study demonstrated the successful construction of a genetic map of a fetus, the approach required an invasive procedure and did not attempt to determine the whole genome sequence of the fetus.

In some embodiments, the methods for predicting a fetal genome sequence may include a step of predicting one or more de novo mutations (i.e., variants occurring only in the genome of the fetus) in a fetal genomic sequence. De novo mutations in the fetal genome should appear within a maternal plasma sequence as ‘rare alleles’ (FIG. 1D), similar to transmitted paternal-specific alleles. However, the detection of de novo mutations poses a much greater challenge: unlike the 1.8×10⁶ paternally heterozygous sites defined by sequencing the father (of which ˜50% are transmitted), the search space for de novo sites is effectively the full genome, throughout which there may be only ˜60 sites given a prior mutation rate estimate of ˜1×10⁻⁸ (Conrad et al. 2011).

As described in the Examples below, such methods for predicting or detecting one or more de novo mutations in a fetal genome sequence may include steps of sequencing (e.g., shotgun sequencing) a paternal genomic sequence from a paternal genomic DNA sample (e.g., cellular genomic DNA in a blood sample or other paternal biological sample), sequencing a maternal genomic sequence from a maternal DNA sample (e.g., cellular genomic DNA in a blood sample or other paternal biological sample), and sequencing one or more maternal-fetal cell-free plasma DNA sequences from a maternal plasma sample. In some embodiments, the paternal genomic sequence and the maternal DNA sequence are then compared to the set of (i.e. one or more, or a plurality of) maternal-fetal cell-free plasma DNA sequences to identify one or more candidate de novo alleles in the fetal genome. Identification of one or more candidate de novo alleles is accomplished by identifying variant alleles which are observed (or “rarely” observed) in the maternal-fetal DNA sequences, but are not observed in the maternal genomic sequence or the paternal genomic sequence.

Once one or more candidate de novo alleles (or a “set” of candidate de novo alleles) are identified, a set of filters are applied to remove known variants and artifacts from sequencing or mapping. Filters that may be applied to the one or more candidate variant alleles may include, but are not limited to, (i) filters that remove known polymorphisms found in NCBI's public database of SNPs (dbSNP, NCBI, see http://www.ncbi.nlm.nih.gov/projects/SNP/) or the 1000 Genome's Pilot 1 database; (ii) filters that remove candidate de novo alleles if the same candidate allele was sequenced with at least moderate base and mapping qualities in another member of the same cohort (i.e., to rule out systematic sequencing errors); (iii) filters that remove candidate de novo alleles with flanking “simple repeat” sequence; and (iv) filters that remove candidate de novo alleles with an excess reads supporting the de novo mutation, relative to the expectation based on the estimated fetal proportion, using a one-tailed binomial test and uncorrected p-value threshold of 0.05. As shown in the Examples below, additional filters defined by one skilled in the art may be used and/or tailored to a certain degree based on a particular dataset. After filters are applied, the candidate variant alleles that remain include at least one true de novo mutation.

In another embodiment, a probability may be assigned to each of the candidate de novo mutations using a method based on a Support Vector Machine (“SVM”). These probabilities may be used to discriminate likely false positives from likely true positives. Although the sensitivity and specificity of this approach are similar to the filter-based approach, this approach is more generalized and should require less fine-tuning on a per-experiment basis.

In some aspects of the methods described in the embodiments herein, a maternal genome sequence, a paternal sequence, or both, are derived from a biological sample which contains genomic DNA. In one aspect, the biological sample is a non-invasive biological sample which contains genomic DNA including, but not limited to, blood and fractions thereof (e.g., plasma, serum), saliva, epithelial cells, bone marrow, and hair. In some embodiments, the maternal sequence is derived from a blood or plasma sample obtained from a female subject during pregnancy. In one aspect, the maternal genome sequence is derived from a maternal plasma sample from a pregnant subject. A maternal blood or plasma sample from a pregnant subject contains the pregnant subject's genomic DNA (i.e., maternal genomic sequence or maternal genome) in circulating cells found in the sample, and also contains a mixture of fetal and maternal DNA in circulating cell-free DNA in the plasma fraction of the blood sample. As such, prediction of maternal inheritance in accordance with the methods described herein may be accomplished with a single sample of maternal blood from a pregnant subject. Further, a maternal blood sample may also be used to identify and/or sequence a fetal genomic sequence or to calculate a percentage fetal DNA in the maternal blood sample. In other aspects, the paternal genome sequence may be derived from a saliva sample or a blood sample. In the case where a haplotype resolved paternal genomic sequence is utilized to predict paternal inheritance, the sample should be of the type which includes sufficient high molecular weight DNA to assemble the haplotype resolved sequence (e.g., a blood sample).

The entire fetal genome is represented in short cfDNA fragments in maternal plasma (Lo et al. 2010). The studies in the Examples below demonstrate the determination of a fetal genome sequence. Substantial completeness and over 99% accuracy may be achieved using a sample of paternal saliva or blood and a single tube of blood collected from the mother at 18.5 weeks gestation. These methods thereby provide an advantage over previous studies (Snyder et al. 2013, which is hereby incorporated by reference as if fully set forth herein).

Only a minority of total cfDNA fragments in maternal plasma is shed from the placenta and thus reflect the fetal inherited complement. For example, the plasma specimens used in the studies in Example 1 below study from two different pregnancies contained 8% and 13% fetoplacental content, which are representative examples given their collection at weeks 8.1 and 18.5, respectively. The remaining cfDNA is derived from maternal cells.

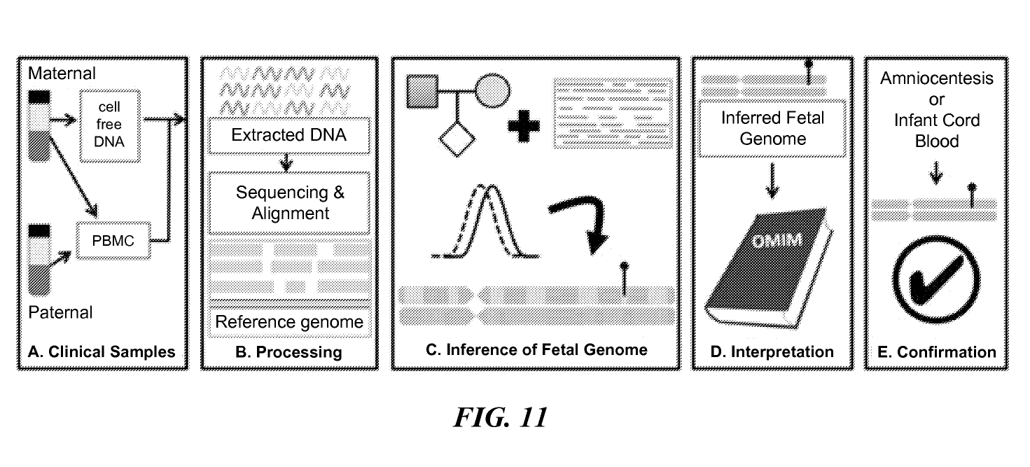

According to the embodiments described herein, deeply sampling this mixture of fetal and maternal genetic material—along with statistical modeling such as that described herein—fetal genotypes can be accurately inferred (FIG. 11). This approach relies on the fact that the fetal genome is necessarily a composite of the parental chromosomes. By determining the parental genotypes, the possible fetal genotypes can generally be constrained on the basis of Mendelian inheritance

In combination with individual and family medical histories, the paternal genotypes establish a set of recessive conditions for which each parent is a carrier. At the majority of sites in the genome (>99.9%), both parents are homozygous for the same allele, and the fetal genotype is therefore unambiguous: homozygous for that allele. At a much smaller proportion of sites (typically fewer than 1×10⁶, or 0.03% of sites, depending upon genetic ancestry), each parent will again be homozygous, but for different alleles; at these sites, the fetus is an obligate heterozygote. Uncertainty about fetal inheritance arises at the remaining sites—those at which one or both parents are heterozygous. The methods described herein address these uncertain sites. According to some aspects of the embodiments described herein, once a suitable biological sample has been obtained, the DNA is isolated and/or extracted, and sequenced to obtain a genomic sequence, which indicates a subject's genotype. In combination with individual and family medical histories, it establishes a set of recessive conditions for which each parent is a carrier. At the majority of sites in the genome (>99.9%), both parents are homozygous for the same allele, and the fetal genotype is therefore unambiguous: homozygous for that allele. At a much smaller proportion of sites (typically fewer than 1×10⁶, or 0.03% of sites, depending upon genetic ancestry), each parent will again be homozygous, but for different alleles; at these sites, the fetus is an obligate heterozygote. Uncertainty about fetal inheritance arises at the remaining sites—those at which one or both parents are heterozygous. The methods described herein address these uncertain cites. To determine the parental genotypes, whole-genome shotgun sequencing (WGS) or any other suitable sequencing technique, is performed on the maternal and paternal genomes. This step may be performed at any time before or during pregnancy.

According to some embodiments, a maternal plasma sample may be used to determining a percentage of fetal DNA in said sample—or the proportion of fetal material among the maternal plasma cfDNA fragments. To estimate the proportion according to some embodiments, a set of informative genetic markers may be identified that would not be observed if the cfDNA were entirely maternal in origin. In one aspect, the homozygous alleles specific to the father (i.e., not carried by the mother) may comprise the set of markers. If the fetus is male, these may be supplemented by sequences specific to the Y chromosome. After deep sequencing of the plasma cfDNA, the frequency of these definitively fetal sequences is tallied, doubled to account for the equal inheritance from the mother, and used as a direct estimate of the percentage of fetal cfDNA in the maternal plasma.

This estimate of the fetal fraction of cfDNA is important for two reasons. First, as this fraction decreases, inaccuracies in the inferred fetal genotypes accumulate. If the fetal cfDNA level is too low—for example, less than 5% —then the accuracy of the predicted fetal genome may drop below 95%, potentially requiring a second plasma sample to be obtained later in pregnancy, when the fetal fraction may be higher. Second, the estimate of fetal concentration is a parameter, along with the parental genotypes and the cfDNA sequencing reads, in a statistical model used to predict fetal inheritance according to the embodiments described herein. As described above, this model is applied to predict the fetal genotypes at the remaining positions of uncertain inheritance: sites at which the mother is heterozygous and could transmit either allele.

In some embodiments, the process of sequencing a genomic sequence or other nucleotide sequence may also include one or more steps including, but not limited to, preparing a library of DNA fragments (e.g., a shotgun library or a DNA fragment library), and amplifying the DNA fragments. The DNA library fragments may be amplified by any suitable method including, but not limited to polony, clone pool dilution, emulsion PCR or bridge PCR. Amplification of the DNA fragments results in the generation of clonal copies or clusters.

Clonal copies or clusters are then sequenced by any suitable sequencing platform or technology for whole genome or targeted sequencing. In some embodiments, suitable sequencing platforms and technologies that may be used in accordance with the methods described herein may include any next generation sequencing or massively parallel sequencing platforms, methods or technologies including, but not limited to, cyclic-array methods, sequencing by hybridization, nanopore sequencing, real-time observation of DNA synthesis, and sequencing by electron microscopy. Suitable applications of DNA sequencing technologies that may be used include, but are not limited to, shotgun sequencing, resequencing, de novo assembly, exome sequencing, DNA-Seq, Targeted DNA-Seq, Methyl-Seq, Targeted methyl-Seq, DNase-Seq, Sono-Seq, FAIRE-seq, MAINE-Seq, RNA-Seq, ChIP-Seq, RIP-Seq, CLIP-Seq, HITS-Seq, FRT-Seq, NET-Seq, Hi-C, Chia-PET, Ribo-Seq, TRAP, PARS, synthetic saturation mutagenesis, Immuno-Seq, Deep protein mutagenesis, PhIT-Seq, SMRT, and genome-wide chromatin interaction mapping. In some embodiments, the methods for capturing contiguity information may be used with “cyclic-array” methods, for applications such as resequencing, de novo assembly, or both as described in detail in International Patent Application Publication No. WO/2012/106546, filed Feb. 2, 2012, which is hereby incorporated by reference as if fully set forth herein.

Suitable DNA sequencing technologies that may be used in accordance with the methods described herein may include, but are not limited to, cyclic-array methods, nanopore sequencing, real-time observation of DNA synthesis, sequencing by electron microscopy. Suitable applications of DNA sequencing technologies that may be used in accordance with the methods described herein may include, but are not limited to resequencing, de novo assembly, exome sequencing, RNA-Seq, ChIP-Seq, and genome-wide chromatin interaction mapping. In some embodiments, the methods for capturing contiguity information may be used with “cyclic-array” methods, for applications such as resequencing, de novo assembly, or both as described in detail in the Examples below. In the embodiments described herein, the haplotype-resolved genome sequencing of a mother, the shotgun genome sequencing or haplotype-resolved genome sequencing of a father, and the deep sequencing of cell-free DNA in maternal plasma may be integrated to predict the whole genome sequence of a fetus (FIG. 1A).

The methods may be used to predict a fetal genome sequence of any length, up to and including a whole genome sequence of the fetus. Thus, in certain embodiments, the methods described herein may be used to predict a whole genome sequence of a fetus, as described in detail in the Examples below.

The methods described above may be used to detect or determine the presence or absence of known, inherited, and/or de novo genetic abnormalities in a fetus. Genetic abnormalities, whether inherited or de novo, may cause or contribute to the development of one or more genetic disorders, congenital abnormalities, specific Mendelian disorders or other diseases or conditions which are linked to one or more genetic abnormalities (e.g., cancer, autoimmune diseases, obesity, heart disease, and inflammatory bowel disease). Thus, in certain embodiments, the methods described herein may be used in a clinical test to screen a fetus for genetic diseases or conditions which are attributable to one or more gene mutations, or to determined the fetus's propensity or risk for developing such diseases or conditions.

Genetic disorders that may be screened for using the methods described herein may include those which are caused by or attributable to SNPs or other genetic abnormalities and include, but are not limited to, Achondroplasia, Alpha-1 Antitrypsin Deficiency, Antiphospholipid Syndrome, Autism, Autosomal Dominant Polycystic Kidney Disease, Breast cancer, Charcot-Marie-Tooth, Colon cancer, Cri du chat, Crohn's Disease, Cystic fibrosis, Dercum Disease, Down Syndrome, Duane Syndrome, Duchenne Muscular Dystrophy, Factor V Leiden Thrombophilia, Familial Hypercholesterolemia, Familial Mediterranean Fever, Fragile X Syndrome, Gaucher Disease, Hemochromatosis, Hemophilia, Holoprosencephaly, Huntington's disease, Klinefelter syndrome, Marfan syndrome, Myotonic Dystrophy, Neurofibromatosis, Noonan Syndrome, Osteogenesis imperfecta, Parkinson's disease, Phenylketonuria, Poland Anomaly, Porphyria, Progeria, Prostate Cancer, Retinitis Pigmentosa, Severe Combined Immunodeficiency (SCID), Sickle cell disease, Skin Cancer, Spinal Muscular Atrophy, Tay-Sachs, Thalassemia, Trimethylaminuria, Turner Syndrome, Velocardiofacial Syndrome, WAGR Syndrome, and Wilson Disease. In other embodiments, the methods described herein may be used to identify new diseases and conditions attributable to genetic abnormalities.

The ability to sequence a fetal genome to high accuracy and completeness will undoubtedly have profound implications for the future of prenatal genetic diagnostics. Although individually rare, when considered collectively, the ˜3,500 Mendelian disorders with a known molecular basis (Amberger et al. 2009)—including those discussed above, contribute substantially to morbidity and mortality (Bell et al. 2011). Currently, routine obstetric practice includes offering a spectrum of screening and diagnostic options to all women. Prenatal screening options have imperfect sensitivity and focus mainly on a small number of specific disorders, including trisomies, major congenital anomalies, and specific Mendelian disorders. Diagnostic tests, generally performed through invasive procedures such as chorionic villus sampling and amniocentesis, also focus on specific disorders and confer risk of pregnancy loss (approximately 0.25-1%). Noninvasive, comprehensive diagnosis of Mendelian disorders early in pregnancy would provide greater amounts of information to expectant parents, without tangible risk.

In certain embodiments, additional methods may be performed to improve the accuracy of the methods for predicting the fetal genome sequence described above. According to one embodiment, such additional methods for improving accuracy may include a reference panel that contains genetic data from unrelated individuals and may be used as a standard for comparison with the predicted fetal genome sequence. Such a comparison may improve prediction accuracy and/or completeness. The process of phasing heterozygous sites yields a set of haplotype blocks and a separate, non-overlapping set of sites that were not phased, due to technical or other reasons, into haplotype blocks. This set of unphased sites includes approximately 10% of genetic variants in each parent. For this subset of unphased sites, predictions of those sites' transmission to the fetus may be made on a site-by-site basis, but the accuracy of these predictions (especially for the maternal genome) is well below the >99% accuracy which was observed for the rest of the genome, for which phase is available.

Therefore, a statistical and computational method was developed to “borrow information” from large panels of unrelated individuals. The method relies on the fact that for any of these identified genetic variants, there is a high probability that the same variant will be observed in another individual. As more and more genomes are sequenced in the field, it becomes increasingly likely that the same variant may be observed in one or more additional sequenced individuals. This possibility is exploited to calculate a scoring metric based in part on linkage disequilibrium between this unphased variant and other nearby variants that have been phased into haplotype blocks, and this score is used to probabilistically assign the previously unphased variant to a haplotype block. This process can be performed with each unphased variant in the maternal and paternal genome. Low-confidence predictions may optionally be discarded by applying thresholds to the per-site scores. When this approach was applied to both parents, about 200,000 additional sites could predicted in the fetal genome with >96% accuracy.

A statistically and computationally similar approach was used to improve accuracy of these predictions. Neighboring haplotype blocks are selected by prioritizing blocks on the basis of the number of heterozygous sites each contains, and selected pairs blocks are merged to make a single, longer block. Again, a scoring metric based on population-based linkage disequilibrium determines whether it is more likely that a given pair of haplotype blocks is joined in one or the other of the two possible ways they could be joined. This approach resulted in a 16% reduction in the prediction error rate.

One impact of this improvement has been to increase the completeness or comprehensiveness of our predictions. Further, this improvement does not confer any additional costs (e.g., no reagent costs or additional experiments to perform) once a population of chromosomes for unrelated individuals has been established as a reference—other than an upfront cost of purchasing a computer suitable for performing such a method. Therefore, this method may be used as an inexpensive and relatively easy way to improve accuracy and completeness of a prediction of a fetal genome sequence.

According to another embodiment for improving the accuracy of predicting may include obtaining and using genetic information (either in the form of whole genome sequencing or other, less expensive methods) from related individuals to improve prediction accuracy and completeness. Additional members of the same family, if available, can be a powerful tool to improve accuracy of experimental phasing of the mother and father. Determining haplotype phase from genotyped individuals in a family pedigree is a standard method in the field, both using genotypes determined by SNP arrays or more recently, by whole-exome or whole-genome sequencing (as an example of the latter, see Dewey F E et al, PLoS Genetics 2011). These additional family members may be used to fix likely errors in the experimental data, or to improve comprehensiveness by phasing a greater proportion of variants. This approach extends and complements the approach outlined above with respect to unrelated individuals.

To demonstrate proof-of-concept for this approach, genomes of several members of a recruited family may be sequenced. For example a recruited family may include, including a mother, father, material from two prior affected fetuses, and maternal plasma from a current, apparently unaffected fetus, and pending delivery of a healthy newborn, cord blood from the offspring. The prior affected fetuses are operationally equivalent to siblings of the current fetus. In addition, this approach may be combined with direct molecular phasing of each parent. This should provide substantial improvement in terms of accuracy and comprehensiveness.

The following examples are intended to illustrate various embodiments of the invention. As such, the specific embodiments discussed are not to be construed as limitations on the scope of the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### EXAMPLES

### Example 1

### Whole Genome Sequencing of a Fetus Using a Haplotype-Resolved Maternal Genome Sequence

The analysis of cell-free fetal DNA in maternal plasma holds great promise for the development of non-invasive prenatal genetic diagnostics. However, studies to date have been largely restricted to the detection of gross abnormalities such as trisomies (Fan et al. 2008; Chiu et al. 2008), the targeted analysis of paternally inherited mutations (Lo & Chiu 2007), or the invasive analysis of a limited number of genomic sites (Lo et al. 2010). As described below, haplotype-resolved genome sequencing (Kitzman et al. 2011) of a mother, shotgun genome sequencing of a father, and deep sequencing of maternal plasma was combined to non-invasively predict the whole genome sequence of a human fetus at 18.5 wks gestation. Inheritance at 1.1×10⁶ phased, maternally heterozygous sites was predicted with 99.3% accuracy, and inheritance at 1.1×10⁶ unphased, paternally heterozygous sites was predicted with 96.6% accuracy. Furthermore, 39 of 44 de novo point mutations in the fetal genome were detected by deep sequencing of maternal plasma. Subsampling of these data, as well as the analysis of a second fetal genome by the same approach, indicate that well-resolved ˜300 kilobase (Kb) parental haplotype blocks combined with relatively shallow sequencing of maternal plasma are sufficient to effectively determine the inherited complement of a fetal genome. However, ultra-deep sequencing of maternal plasma is necessary for sensitively detecting de novo mutations in the fetus at a genome-wide scale. The non-invasive analysis of inherited and de novo variation in fetal genomes should facilitate the comprehensive prenatal diagnosis of both recessive and de novo dominant Mendelian disorders.

### Materials and Methods

Subjects. Two mother-father-child trios were recruited for the studies described below: “I1”—a first trio at 18.5 wks gestation (the “I1 trio”); and “G1”—a second trio at 8.2 wks gestation (the“G1 trio”). Tables 1 and 2 show individuals sequenced, type of starting material, and final fold-coverage of the reference genome after discarding PCR or optical duplicate reads for the I1 trio and the G1 trio, respectfully (GA, gestational age). The Examples below focus primarily on the results of “I1,” the trio for which considerably more sequence data of each type was generated.

TABLE 1
Subject information for the I1 Trio

| Individual | Sample | Depth of coverage |
|---|---|---|
| Mother (I1-M) | Plasma (5 ml, GA 18.5 weeks) | 78 |
| | Whole Blood (<1 mL) | 32 |
| Father (I1-P) | Saliva | 39 |
| Offspring (I1-C) | Cord blood at delivery | 40 |

TABLE 2
Subject information for the G1 Trio

| Individual | Sample | Depth of coverage |
|---|---|---|
| Mother (G1-M) | Plasma (4 ml, GA 8.14 weeks) | 56 |
| | Whole Blood | 25 |
| Father (G1-P) | Whole Blood | 33 |
| Offspring (G1-C) | Cord blood at delivery | 44 |

Whole-Genome Shotgun Library Preparation and Sequencing.

Genomic DNA (maternal and/or paternal) was extracted from whole blood, as available, or alternatively from saliva, with the Gentra Puregene Kit (Qiagen), or OrageneDx (DNA genotek), respectively. Purified DNA was fragmented by sonication with a Covaris S2 instrument (Covaris). Indexed shotgun sequencing libraries were prepared using the Kapa Library Preparation Kit (Kapa Biosystems), following the manufacturer's instructions. All libraries were sequenced on Hiseq 2000 instruments (Illumina) using paired-end 101 bp reads with an index read of 9 bp.

Maternal Plasma Library Preparation and Sequencing.

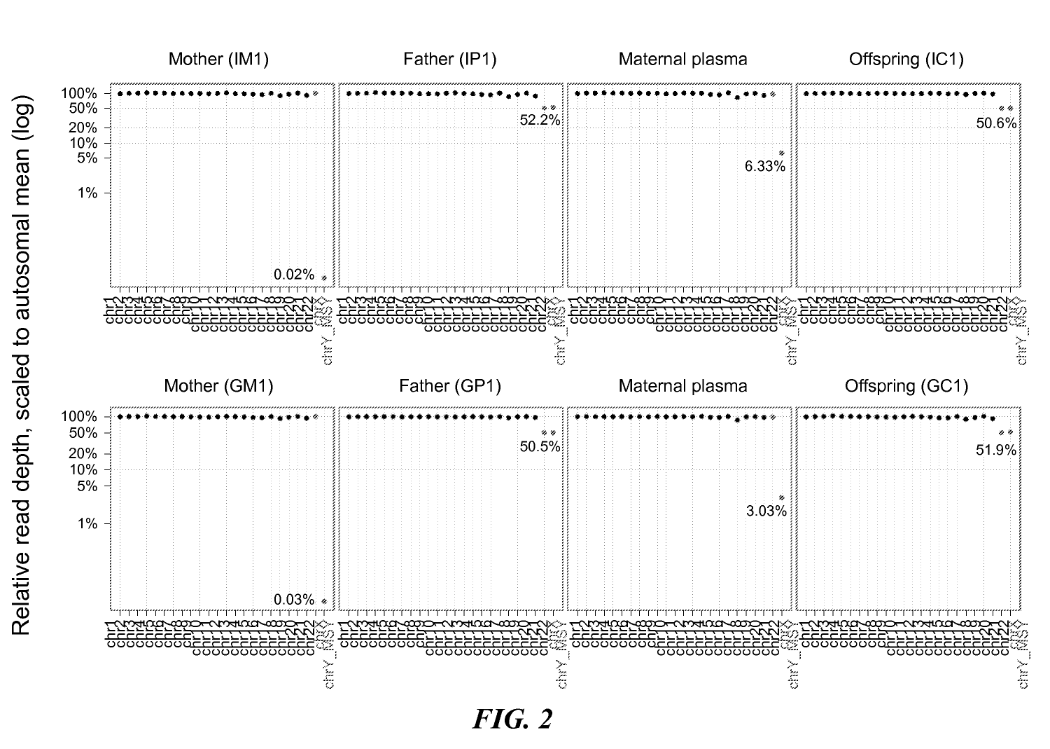

Maternal plasma was collected by standard methods and split into 1 ml aliquots which were individually purified with the Qiaamp Circulating Nucleic Acids kit (Qiagen). DNA yield was measured with a Qubit fluorometer (Invitrogen). Sequencing libraries were prepared with the ThruPlex-FD kit (Rubicon Genomics), which includes a proprietary series of end-repair, ligation, and amplification reactions. Index read sequencing primers compatible with the WGS and fosmid libraries from this study were included during sequencing of maternal plasma libraries to permit detection of any contamination from other libraries. The percentage of fetal-derived sequences was estimated from plasma sequences by counting alleles specific to each parent as well as sequences mapping specifically to the Y chromosome (FIG. 2, Lo et al. 1999).

Maternal haplotype resolution via clone pool dilution sequencing. Haplotype-resolved genome sequencing was performed as previously described (Kitzman et al. 2011), with minor updates to facilitate processing in a 96-well format. Briefly, high molecular weight DNA was mechanically sheared to mean size ˜38 kbp using a Hydroshear instrument (DigiLab), with the following settings: volume=120 ul, cycles=20, speed code=16. Sheared DNA was electrophoresed through 1% Low Melting Point UltraPure agarose (Invitrogen) with the buffer (0.5×TBE) chilled to 16° C., using the following settings on a BioRad ChefDR-II pulsed field instrument: 170V, initial A=1, final A=6. After running for 17 h, lanes containing size markers (1 kbp extension ladder, Invitrogen) were excised, stained with SYBR Gold dye (Invitrogen), and placed alongside the unstained portion of the gel on a blue light transilluminator. The band between 38-40 kbp was then excised, melted for 10 min in a 70° C. water bath, spun at 15,000 rpm to pellet debris, and incubated at 47° C. for 1 h with 0.5 units beta-agarase (Promega) per 200 mg gel to digest the agarose. Sheared, size-selected DNA was precipitated onto Ampure XP beads (Beckman Coulter) as follows: 100 ul of beads in the supplied buffer were supplemented with additional binding buffer (2.5M NaCl+20% PEG 8000) to match the volume of the digested gel and DNA. The beads and buffer were then gently mixed with the DNA/agarase reaction mixture, pelleted and rinsed following the manufacturer's directions, and finally eluted into 60 ul H2O. DNA was next end-repaired with the End-IT kit (Epicentre), cleaned up by precipitation onto 30 ul Ampure XP beads supplemented with 70 ul additional binding buffer, and eluted into 12 ul H2O. Ligation to the fosmid vector backbone pCC1 Fos and clone packaging were conducted as previously described using the CopyControl Fosmid Construction Kit (Epicentre). A single bulk infection per maternal sample was performed using each phage library and each was then split by dilution into 1.5 ml cultures (LB+12.5 ug/ml chloramphenicol) across a deep-well 96-well plate. The resulting master culture was grown overnight at 37° C. shaking at 225 rpm. The following day, subcultures were made by in 96-well plates by adding 200 ul inoculum from each master culture well into fresh outgrowth media (LB+12.5 ug/ml chloramphenicol+1× final autoinduction solution) to a final volume of 1.5 ml per well. After overnight outgrowth (37° C., 225 rpm shaking), clone pool DNA was extracted by alkaline lysis mini-preparation in 96 well plates, following standard procedures (J. Sambrook, Molecular Cloning: A Laboratory Manual, Third Edition (3 volume set) (Cold Spring Harbor Laboratory Press, ed. 3, 2001), p. 2344, which is hereby incorporated by reference as if fully set forth herein). Indexed Illumina sequencing libraries were prepared in sets of 96 using the Nextera library preparation kit as previously described (Adey et al. 2010), followed by library pooling and size selection to 350-650 bp.

Variant Calling.

Reads were split by index, allowing up to edit distance of 3 to the known barcode sequences, and then mapped to the human reference genome sequence (hg19) using bwa v0.6.1 (Li et al. 2009). After removing PCR duplicate read pairs using the Picard toolkit (http://picard.sourceforge.net/), local realignment around indels, variant discovery, quality score recalibration and filtering to 99% estimated sensitivity among known polymorphisms was performed using the Genome Analysis Toolkit (DePristo et al. 2011) using “best practices” parameters provided by the software package's authors (http://www.broadinstitute.org/gsa/wiki/).

Haplotype Assembly.

Reads were split per dilution pool by barcode, and a sliding-window read depth measure was used to infer clone positions (Kitzman et al. 2011). Using custom scripts, clone pool reads were re-genotyped against heterozygous SNPs ascertained by shotgun sequencing, and overlapping clones from different pools were assembled into haplotype blocks with a modified HapCUT algorithm (Bansal & Bafna 2009).

Inference of the Fetal Genome Sequences.

A Hidden Markov model (HMM) was constructed to infer the inherited maternal allele at each maternal-specific heterozygous site. The model's latent state defines which of the two phased maternal haplotype blocks is inherited at each site, with a third state representing a between block region at which phase is unknown. The HMM emits allele counts at each phased site, with probabilities given by binomial distribution parameterized as follows: if the maternally inherited allele is identical to the paternal (homozygous) allele at a given “maternal-only” heterozygous site, the probability of observing k such alleles among N total reads with fetal percentage F is

$$Pr ( K = k \vert N , F ) = Bin ( N , \frac{1 - F}{2} + \frac{F}{2} + \frac{F}{2} ) \qquad ( Equation 1 )$$

where the first term in the second binomial parameter represents the expected allele balance in the maternally-derived DNA in the maternal plasma, the second term represents the expected contribution of the paternal allele via the fetus, and the third term represents the expected contribution of the inherited maternal allele via the fetus.

If the inherited maternal allele and the paternal allele differ at a given site, the probability of observing k inherited maternal alleles simplifies to

$$Pr ( K = k \vert N , F ) = Bin ( N , \frac{1 - F}{2} + \frac{F}{2} ) = Bin ( N , 0.5 ) \qquad ( Equation 2 )$$

Inferred transitions within phased blocks represent either true recombination events or switch errors in maternal phasing. Transition probabilities within phased blocks were held constant at 10⁻⁵; changing this parameter did not significantly affect either the number of inferred transitions within blocks or the final accuracy. Finally, the most probable path through the observed data was determined using the Viterbi algorithm for inference of the latent state at each site, corresponding to a prediction of the inherited maternal allele. Prediction accuracy was determined by comparing the predicted to actual inheritance determined from the offspring's genotype.

Inheritance at “paternal-only” heterozygous sites was predicted using a binomial model. At each such site, either the paternal-specific allele or the allele shared with the mother can be transmitted. Let F represent the fetal DNA concentration in the maternal plasma and N represent the depth at a given site. If the paternal-specific allele is transmitted, the allele should be observed in N×F/2 times in the maternal plasma. Similarly, if the paternal-specific allele is not transmitted, the allele should be observed 0 times. The likelihoods of observing K such alleles from N total under each inheritance models were compared, and prediction was determined by choosing the model that yielded a higher likelihood.

At each shared heterozygous site (i.e., heterozygous in both parents), the maternally contributed allele was predicted based on the inferred inheritance of the block in which the site is situated, as determined by “maternal-only” heterozygous sites within the same block. In the rare event that a block was identified to be partially inherited, either due to a real recombination event or a switch error in phasing, the inferred inheritance of the nearest “maternal-only” heterozygous site within the block was used to assign a prediction.

Downsampling Methodology.

The effect of reduced fetal contribution to the maternal plasma sequences was investigated by diluting the fetal-specific sequences in silico and reanalyzing the modified data. Simulated dilution of fetal content was carried out as follows. At each maternal-specific heterozygous site, alleles A and B were observed with counts N_{A} and N_{B} among the full dataset, with N_{TOTAL}=N_{A}+N_{B}. For a given dilution coefficient D/F where 0<D<F, the total pool of observed counts was diluted by first increasing N_{TOTAL} by a factor of F/D, with additional counts allocated by assigning each new allele randomly to N_{A} or N_{B} with equal probability, and then sampling counts from the temporarily expanded pool by discarding each allele from N_{A} and N_{B} with probability 1−D/F. Updated counts and fetal content estimates were used as input into the Hidden Markov model described above. Reduced coverage within plasma data was separately simulated by subsampling a portion of the observed counts at each site. For a given proportion S, each observed base was discarded with probability 1−S. Updated counts were then used as input into the Hidden Markov model as described.

### Results

Genome Sequencing

In brief, the haplotype-resolved genome sequence of the mother (“I1-M”) was determined by first performing shotgun sequencing of maternal genomic DNA from blood to 32-fold coverage (coverage=median-fold coverage of mapping reads to the reference genome after discarding duplicates). Next, by sequencing complex haploid subsets of maternal genomic DNA while preserving long-range contiguity (Kitzman et al. 2011), 91.4% of 1.9×10⁶ heterozygous SNPs into long haplotype blocks (N50 of 326 kilobases (kbp)). The shotgun genome sequence of the father (“I1-P”) was determined by sequencing of paternal genomic DNA to 39-fold coverage, yielding 1.8×10⁶ heterozygous SNPs. However, paternal haplotypes could not be assessed because only relatively low molecular weight DNA obtained from saliva was available. Shotgun DNA sequencing libraries were also constructed from 5 mL of maternal plasma (obtained at 18.5 wks gestation), and this “genome” (a mixture of maternal- and fetal-derived cell-free DNA) was sequenced to 78-fold non-duplicate coverage. The fetus was male, and fetal content in these libraries was estimated at 13%. To properly assess the accuracy of the methods for determining the fetal genome solely from samples obtained non-invasively at 18.5 wks gestation, shotgun genome sequencing of the child (“I1-C”) was also performed to 40-fold coverage via cord blood DNA obtained after birth.

### Maternal-Only Heterozygous Transmission

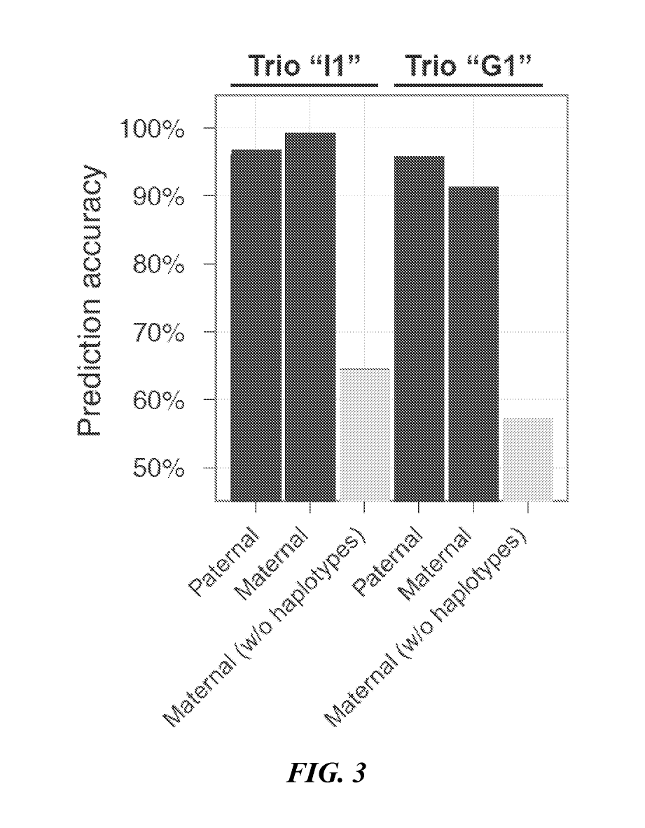

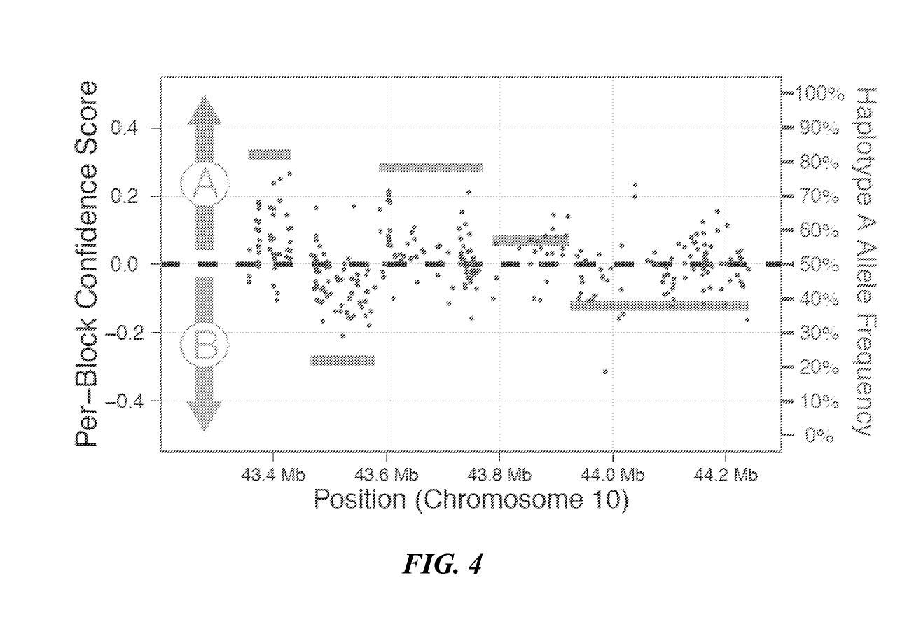

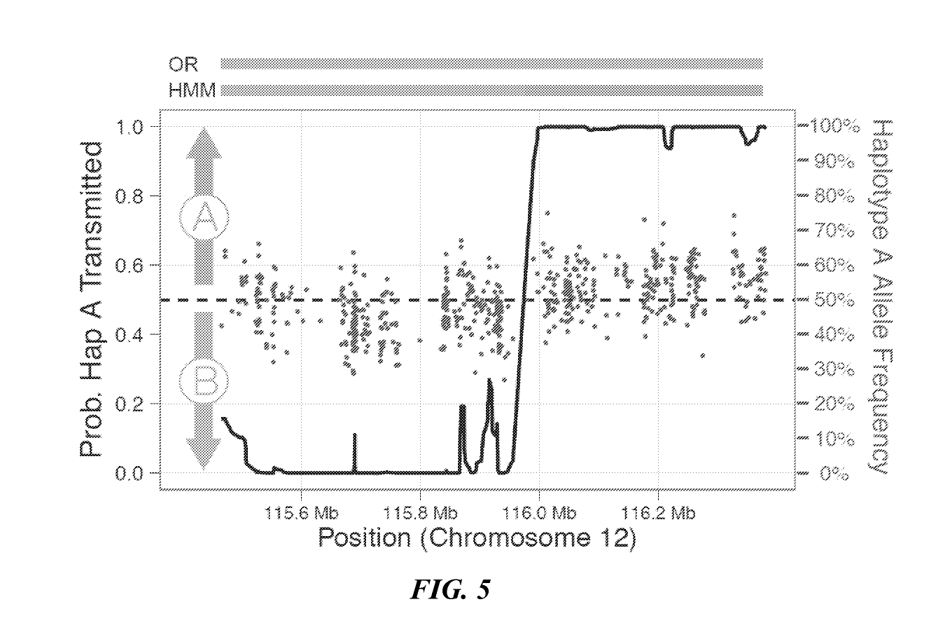

In some embodiments, the methods described herein may include a step of predicting transmission at ‘maternal-only’ heterozygous sites. Given the fetal-derived proportion of ˜13% in cell-free DNA, the maternal-specific allele is expected in 50% of reads aligned to such a site if it is transmitted, versus 43.5% if the allele shared with the father is transmitted. However, even with 78-fold coverage of the maternal plasma “genome”, the variability of sampling is such that site-by-site prediction results in only 64.4% accuracy (FIG. 3). Therefore, the allelic imbalance was examined across blocks of maternally heterozygous sites defined by haplotype-resolved genome sequencing of the mother (FIG. 1B). The vast majority of experimentally defined maternal haplotype blocks having a haplotype assembly N50 of 326 Kb were wholly transmitted, with partial inheritance in a small minority of blocks (0.6%, n=72) corresponding to switch errors from haplotype assembly and to sites of recombination. A Hidden Markov model (HMM) was developed to identify likely switch sites and thus more accurately infer the inherited alleles at maternally heterozygous sites (FIG. 4, FIG. 5). Using this model, accuracy of the inferred inherited alleles at 1.1×10⁶ phased, ‘maternal-only’heterozygous sites increased from 98.6% to 99.3% (Table 3, 11 Trio). Sites later determined by trio sequencing (including the offspring) to have poor genotype quality scores or genotypes that violated Mendelian inheritance were discarded the purpose of evaluating accuracy (14,000 maternal-only, 32,233 paternal-only, and 480 shared heterozygous sites, or 1.5% of all sites). Among biparentally heterozygous sites, accuracy was assessed only where the offspring was homozygous (48.8%, n=631,721), allowing the “true” transmitted alleles to be unambiguously inferred from trio genotypes. Results from G1 Trio are shown in Table 4.

TABLE 3
Number of sites and accuracy of fetal genotype inference from maternal
plasma sequencing for I1 trio

| | | Other parental | Number of | |
|---|---|---|---|---|
| Individual | Site | genotype | sites | Accuracy |
| Mother | Heterozygous, | Homozygous | 1,064,255 | 99.3% |
| | phased | Heterozygous | 576,242 | 98.7% |
| | Heterozygous, | all | 121,425 | N.D. |
| | not phased | | | |
| Father | Heterozygous | Homozygous | 1,134,192 | 96.8% |
| | | Homozygous | 631,721 | N.D. |

TABLE 4
Number of sites and accuracy of fetal genotype inference from maternal
plasma sequencing for G1 trio

| | | Other parental | Number of | |
|---|---|---|---|---|
| Individual | Site | genotype | sites | Accuracy |
| Mother | Heterozygous, | Homozygous | 1,141,600 | 95.7% |
| | phased | Heterozygous | 683,669 | 91.3% |
| | Heterozygous, | all | 102,534 | N.D. |
| | not phased | | | |
| Father | Heterozygous | Homozygous | 1,062,805 | 60.3% |
| | | Homozygous | 771,987 | N.D. |

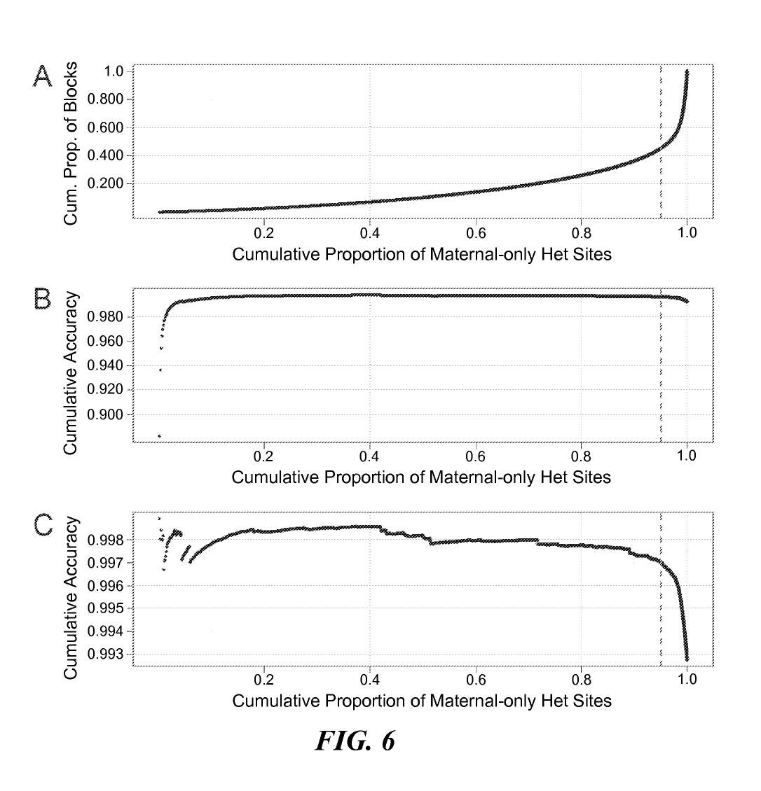

Remaining errors were concentrated among the shortest maternal haplotype blocks (FIG. 6), which provide less power to detect allelic imbalance in plasma data as compared with long blocks. Among the top 95% of sites ranked by haplotype block length, prediction accuracy rose to 99.7%, suggesting that remaining inaccuracies can be mitigated by improvements in haplotyping.

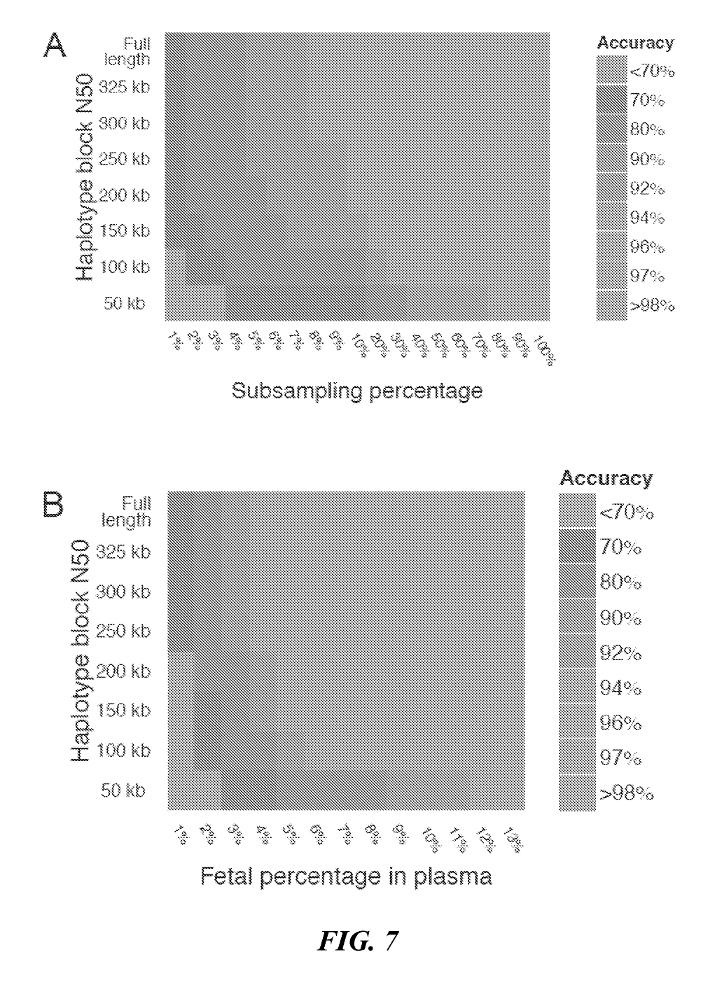

Simulations were performed to characterize how the accuracy of haplotype-based fetal genotype inference depended upon haplotype block length, maternal plasma sequencing depth, and the fraction of fetal-derived DNA. To mimic the effect of less successful phasing, maternal haplotype blocks were split into smaller fragments to create a series of assemblies with decreasing contiguity. A range of sequencing depths were then subsampled from the pool of observed alleles in maternal plasma, and predicted the maternally contributed allele at each site as above (FIG. 7A). The results suggest that inference of the inherited allele is robust to either decreasing sequencing depth of maternal plasma, or to shorter haplotype blocks, but not both. For example, using only 10% of the plasma sequence data (median depth=8×) in conjunction with full-length haplotype blocks, inheritance at 94.9% of ‘maternal-only’ heterozygous sites were successfully predicted. A nearly identical accuracy (94.8%) was achieved at these sites when highly fragmented haplotype blocks (N50=50 Kb) were used with the full set of plasma sequences. Next, decreased proportions of fetal DNA were simulated in the maternal plasma by spiking in additional depth of both maternal alleles at each site and subsampling from these pools, effectively diluting away the signal of allelic imbalance used as a signature of inheritance (FIG. 7B). Again, the accuracy of the model was found to be robust to either lower fetal DNA concentrations or shorter haplotype blocks, but not both.

Paternal-Only Heterozygous Transmission

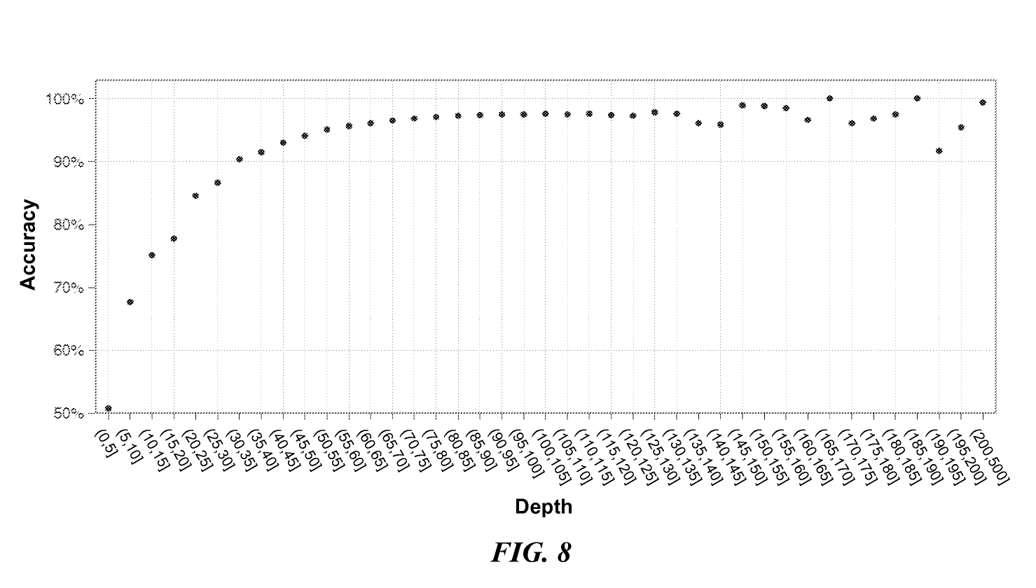

The methods described herein include predicting transmission at ‘paternal-only’ heterozygous sites. At these sites, when the father transmits the shared allele, the paternal-specific allele should be entirely absent among the fetal-derived sequences. If instead the paternal-specific allele is transmitted, it will on average constitute half the fetal-derived reads within the maternal plasma “genome” (˜5 reads given 78-fold coverage, assuming 13% fetal content). To assess these, a site-by-site log-odds test was performed; this amounted to taking the observation of one or more reads matching the paternal-specific allele at a given site as evidence of its transmission, and conversely the lack of such observations as evidence of non-transmission (FIG. 1C). In contrast to maternal-only heterozygous sites, this simple site-by-site model was sufficient to correctly predict inheritance at 1.1×10⁶ paternal-only heterozygous sites with 96.8% accuracy (Table 3, above). Accuracy may be improved upon by deeper sequence coverage of the maternal plasma “genome” (FIG. 8), or alternatively by taking a haplotype-based approach using a high molecular weight genomic DNA sample from the father (i.e., a blood or plasma sample).

Maternal and Paternal (or “Parental”) Heterozygous Transmission

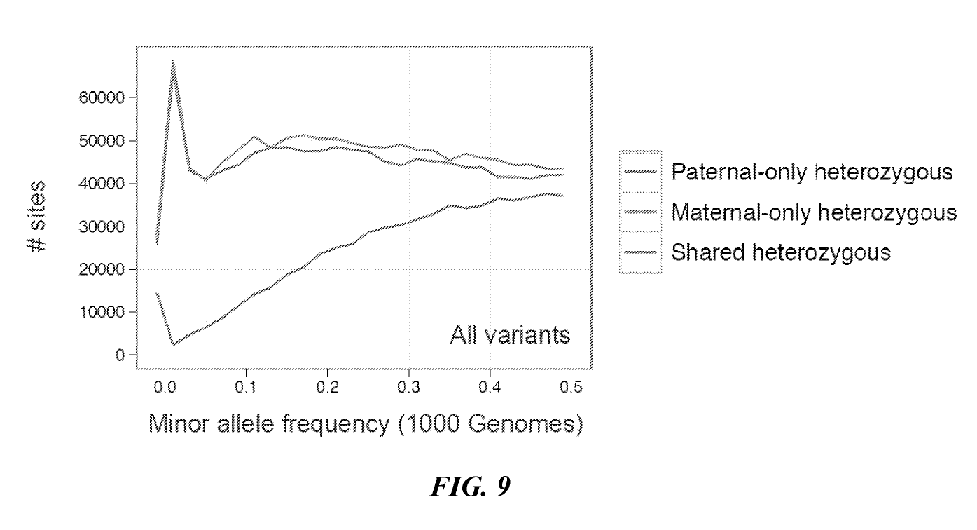

The methods described herein and the study described above may include a step of predicting transmission at variant allelic sites that are heterozygous in both parents. Maternal transmission at such shared sites phased using neighboring ‘maternal-only’ heterozygous sites were predicted in the same haplotype block. This yielded predictions at 576,242/631,721 (91.2%) of shared heterozygous sites with an estimated accuracy of 98.7% (Table 3, above). Although paternal transmission was not predicted at these sites, this could be done with high accuracy given paternal haplotypes, analogous to the case of maternal transmission described above. It was noted that shared heterozygous sites primarily correspond to common alleles (FIG. 9), which are less likely to contribute to Mendelian disorders in non-consanguineous populations.

### Discussion

As described above, noninvasive prediction of the whole genome sequence of a human fetus was demonstrated through the combination of haplotype-resolved genome sequencing (Kitzman et al. 2011) of a mother, shotgun genome sequencing of a father, and deep sequencing of maternal plasma. Of note, the types and quantities of materials used were consistent with those routinely collected in a clinical setting (see Tables 1 (above) and 5 (below)). To replicate these results, the full experiment was repeated for a second trio (“G1”) from which maternal plasma was collected earlier in the pregnancy, at 8.2 weeks after conception. Both the overall sequencing depth and the fetal-derived proportion were each lower relative to the first trio (by 28% and 51%, respectively), resulting in an average of fewer than four fetal-derived reads per site. Nevertheless, a 95.7% accuracy was achieved for prediction of inheritance at maternal-only sites, consistent with accuracy obtained under simulation with data from the first trio (FIG. 7). These results underscore the importance of specific technical parameters in determining performance, namely the length and completeness of haplotype-resolved sequencing of parental DNA, and the depth and complexity of sequencing libraries derived from low starting masses of plasma-derived DNA (less than 5 ng for both 11 and G1 in the study).

The analyses described herein focus on single nucleotide variants, which are the most common form of both non-pathogenic and pathogenic genetic variation in human genomes (Durbin 2010; Stenson et al. 2009). Clinical application of non-invasive fetal genome sequencing may include additional methods for detecting other forms of variation, e.g. insertion-deletions, copy number changes, and structural rearrangements. Techniques for the detection of other forms of variation may derive from short sequencing reads in a manner that is directly integrated with experimental methods and algorithms for haplotype-resolved genome sequencing.

### Example 2

### Inference of Paternal Inheritance Using a Haplotype-Resolved Paternal Genome

In addition to those methods described above, the following methods were implemented to infer paternal inheritance of a genetic abnormality.

Hidden Markov Model for Prediction of Paternal Inheritance.

An HMM was constructed to infer the inherited paternal allele at each paternal-specific heterozygous site. The model's latent state defines which of the two phased paternal haplotype blocks is inherited at each site, with a third state representing a between-block region at which phase is unknown. The HMM emits allele counts at each phased site, with emission probabilities given by binomial distribution parameterized as follows: If the paternally inherited allele is identical to the maternal (homozygous) allele at a given paternal-only heterozygous site, the probability of observing k such alleles among N total reads with fetal percentage F is given by:

Pr(K=k|N,F)−Bin(N,1−ε) (Equation 3)

where ε is a small number representing the probability of a sequencing or technical error.

If the inherited paternal allele and the homozygous maternal allele differ at a given site, the probability of observing k copies of the inherited paternal allele is given by:

$$Pr ( K - k \vert N , F ) - Bin ( N , \frac{F}{2} ) \qquad ( Equation 4 )$$

As in the maternal model described above, the Viterbi algorithm was to evaluate the most probable path, and held the transition probabilities constant at 10⁻⁵.

### Example 3

### Detection of De Novo Mutations in a Fetal Genome

In addition to those methods described above, the following methods were implemented

Determination of Candidate De Novo Mutations.

As described above, the shotgun sequenced paternal genomic sequence and the maternal DNA sequence were compared to the maternal-fetal cell-free plasma DNA sequences to identify one or more candidate variant alleles in the fetal genome. Identification of one or more candidate de novo alleles is accomplished by identifying variant alleles which are observed (or “rarely” observed) in the maternal-fetal DNA sample (i.e., in maternal plasma), but are not observed in the maternal genomic sequence or the paternal genomic sequence (FIG. 1D).

Filters for De Novo Candidate Set.

Starting with all sites identified as heterozygous in the offspring and homozygous in a reference (or corresponding) sequence in both parent (i.e., those sites observed in the fetal DNA, but not in the maternal or paternal DNA), a set of filters were applied to the candidate set of candidate de novo mutations. The following filters were then applied to the set of candidate de novo mutations to identify one or more true de novo mutations:
1. Remove known variants (variants in the dbSNP database (v135) or 1000 Genomes Pilot 1 database)
2. Remove candidates with low coverage in one or both parents (i.e., sites where the parental genotypes are less confident). This was defined as <15 reads for the 11 trio, and <10 reads for the G1 trio)
3. Remove candidate de novo alleles if the same candidate allele was sequenced with at least moderate base and mapping qualities in another member of the same cohort (i.e., to rule out systematic sequencing errors, described above).
4. Remove candidates with variant quality score below 230.
5. Remove candidates supported only by a set of reads with fewer than three distinct alignment endpoints.
6. Remove candidates supported by fewer than two high-quality reads.
7. Remove candidates with flanking “simple repeat” sequence.
8. Remove candidates with an excess of reads supporting the de novo mutation, relative to the expectation based on the estimated fetal proportion, using a one-tailed binomial test and uncorrected p-value threshold of 0.05. In the current example, Phred-scaled base quality >=10 and mapping quality >=20)
9. Remove candidates supported only by a set of Phred-scaled base qualities whose sum was less than or equal to 105.

De novo mutations were validated by PCR and direct capillary sequencing (see Table 5, Table 6, and Table 7). Briefly, each event in the G1 and 11 Trio was targeted for validation by PCR and direct capillary sequencing. As shown in Table S1 below, amplification and sequencing succeed at 35 of 44 sites; of those, all 35 validated as true de novo point mutations (i.e., offspring heterozygous and parents homozygous for reference allele).

TABLE 5
De novo point mutations identified by whole-genome shotgun
sequencing in two trios (G1 and 11).

| | | SEQ ID |
|---|---|---|
| Primer Set | Sequence | NO: |
| ACMSD de novo | tgtaaaacgacggccagtACTGACTGCTGCCTGAAGGT/ | 1 |
| mutation | caggaaacagctatgacCCCCACCAAAGCAGATAAAC | 2 |
| validation | | |
| chr1_14827232 | tgtaaaacgacggccagtACTCCAAGCAAGCAGAAGGA | 3 |
| chr1_14827232 | caggaaacagctatgacCCAGGAATTTTCCCATTTCA | 4 |
| chr1_21959596 | tgtaaaacgacggccagtCAGATGCCTTCCTAGGGTGA | 5 |
| chr1_21959596 | caggaaacagctatgacGGTATGAGGTTGAGGCTGGA | 6 |
| chr1_62642578 | tgtaaaacgacggccagtGATGCACCAGGTTCCCTAGA | 7 |
| chr1_62642578 | caggaaacagctatgacGTGCCTGAATTCCAAAAGGA | 8 |
| chr1_158061739 | tgtaaaacgacggccagtGGCTACTCCCCTCTGATTCC | 9 |
| chr1_158061739 | caggaaacagctatgacATGGGCGTGTTATTCCCTTT | 10 |
| chr1_176538426 | tgtaaaacgacggccagtCACACAAACTTGCACATCCA | 11 |
| chr1_176538426 | caggaaacagctatgacCAATTCAGGTGCATGTGGTT | 12 |
| chr1_197602948 | tgtaaaacgacggccagtCCTCAGCATTCCCCTACCTT | 13 |
| chr1_197602948 | caggaaacagctatgacTGCTGGAAAGCCATATGAGA | 14 |
| chr2_32296201 | tgtaaaacgacggccagtTTGGAAGACTGAAAACCTGTGA | 15 |
| chr2_32296201 | caggaaacagctatgacCCTCCACCATTGTGTTACCC | 16 |
| chr2_58060266 | tgtaaaacgacggccagtTGAAAATGGCCATACTGCCTA | 17 |
| chr2_58060266 | caggaaacagctatgacTAAACCATGCAAATGCTCCA | 18 |
| chr2_190042167 | tgtaaaacgacggccagtGCTTTTCATGTGTGCCTCAG | 19 |
| chr2_190042167 | caggaaacagctatgacGCAAAAGAAAGTCGTCATTGC | 20 |
| chr2_238760708 | tgtaaaacgacggccagtCCAGAGAAATGGCATTGTGA | 21 |
| chr2_238760708 | caggaaacagctatgacAGGCAGGAGAATTGCTTGAA | 22 |
| chr3_17614899 | tgtaaaacgacggccagtAGGGATGGCATTATCAACCA | 23 |
| chr3_17614899 | caggaaacagctatgacTTCAAATTGCAGTGCTAGGC | 24 |
| chr3_18023875 | tgtaaaacgacggccagtCCCCAAAATGTGAAATTGCT | 25 |
| chr3_18023875 | caggaaacagctatgacTAGCCAATTGGTAGGCTGGT | 26 |
| chr3_36828198 | tgtaaaacgacggccagtCTGGGTGCTGTTTATGTGGA | 27 |
| chr3_36828198 | caggaaacagctatgacCCTGAGATGGGAAGGAATGA | 28 |
| chr3_79639506 | tgtaaaacgacggccagtTGCTTTGATTTCCCACCAAT | 29 |
| chr3_79639506 | caggaaacagctatgacGATCAATTCTCCCTGGCAAA | 30 |
| chr3_167679627 | tgtaaaacgacggccagtAGGACAGCCATGACTTAGGC | 31 |
| chr3_167679627 | caggaaacagctatgacGCCCTCCTGAATGGGATTAG | 32 |
| chr3_188400668 | tgtaaaacgacggccagtCTCTTCAGGCCCTAATGCAC | 33 |
| chr3_188400668 | caggaaacagctatgacCATTGCCACAGAGTCGGTAA | 34 |
| chr4_28535313 | tgtaaaacgacggccagtCCGTGTTTAGCCAGAATGGT | 35 |
| chr4_28535313 | caggaaacagctatgacGGTTTTTCCACTTGCTTTCG | 36 |
| chr4_32286182 | tgtaaaacgacggccagtCAGTTCTCCGCACACTGATG | 37 |
| chr4_32286182 | caggaaacagctatgacGACCCTCATGGCAGTCTATTTC | 38 |
| chr4_38294675 | tgtaaaacgacggccagtTGAGTCATGCTGGAAATGGA | 39 |
| chr4_38294675 | caggaaacagctatgacATTCCAAAGCTTCTCCCACA | 40 |
| chr5_5059500 t | gtaaaacgacggccagtCAGCATAAAGGTGGTTTGGAA | 41 |
| chr5_5059500 c | aggaaacagctatgacTCCCATTGTGTTTGGCTACA | 42 |
| chr5_19601463 | tgtaaaacgacggccagtGCAGGAGGTTACAAGCCAGT | 43 |
| chr5_19601463 | caggaaacagctatgacTCTGTTGCAGGGCTTTTCTT | 44 |
| chr5_38960933 | tgtaaaacgacggccagtCCATTGGGATAAATGGCAAG | 45 |
| chr5_38960933 | caggaaacagctatgacATTGTGGCAGAAGGGGAAG | 46 |
| chr5_133747799 | tgtaaaacgacggccagtCCAGGGCTACAAGGGTCTTT | 47 |
| chr5_133747799 | caggaaacagctatgacTGTGCCTGGCAAGTATTCAC | 48 |
| chr6_63446051 | tgtaaaacgacggccagtTTGCTTCAGGCTTCTCTTCC | 49 |
| chr6_63446051 | caggaaacagctatgacGCGTGTTGTTTAAGCCTCCT | 50 |
| chr7_77442453 | tgtaaaacgacggccagtCCAGGCCTTCAAGCATTTTA | 51 |
| chr7_77442453 | caggaaacagctatgacTCGCTAAATGCAATGGTCAG | 52 |
| chr7_85735259 | tgtaaaacgacggccagtAGTGGGGAAGATGGAAGGAG | 53 |
| chr7_85735259 | caggaaacagctatgacATTTTGGTGGGGACACAGAG | 54 |
| chr9_18393440 | tgtaaaacgacggccagtTGTTGCTGTTGCATTCCACT | 55 |
| chr9_18393440 | caggaaacagctatgacCCTCAATGACCACCACACTG | 56 |
| chr9_31764904 | tgtaaaacgacggccagtTAAGTCCGAAACCCAACAGG | 57 |
| chr9_31764904 | caggaaacagctatgacCCAATGGGACACTGCCTAGT | 58 |
| chr9_36929059 | tgtaaaacgacggccagtGCTTTGACTGCCAGGAAACT | 59 |
| chr9_36929059 | caggaaacagctatgacTTCCTTCCTTCCTTCCTTCC | 60 |
| chr9_38730375 | tgtaaaacgacggccagtTTAACAGGTGTGAGCCACCA | 61 |
| chr9_38730375 | caggaaacagctatgacGCCTTCTTCAACCACACACA | 62 |
| chr10_92799212 | tgtaaaacgacggccagtTTAGGCCAGAGATGCTTGCT | 63 |
| chr10_92799212 | caggaaacagctatgacCCCACTCCTCCCACTCCTAT | 64 |
| chr11_18977014 | tgtaaaacgacggccagtGGAACTGGTGGGGCATATTT | 65 |
| chr11_18977014 | caggaaacagctatgacCCACAGTTGGAAGCTCGATT | 66 |
| chr11_74729193 | tgtaaaacgacggccagtACTGGGAGGTAGGGAGGAAA | 67 |
| chr11_74729193 | caggaaacagctatgacTGAGAGTTGTTGTGCCCACT | 68 |
| chr13_43751146 | tgtaaaacgacggccagtTCAGAAGGAGCCAAATCAGG | 69 |
| chr13_43751146 | caggaaacagctatgacGGTTCAGTCAAAGCCAAAGC | 70 |
| chr14_27178898 | tgtaaaacgacggccagtGCTTCAGATTGTTTCTTCCACA | 71 |
| chr14_27178898 | caggaaacagctatgacGATTCCAATGTGAGGGCAAG | 72 |
| chr14_38414572 | tgtaaaacgacggccagtTACCATGGGACTCTGGAAGC | 73 |
| chr14_38414572 | caggaaacagctatgacCCAACAGACGAAGGATTGCT | 74 |
| chr15_59850650 | tgtaaaacgacggccagtAGGTTGCAGTGAGCCAAGAT | 75 |
| chr15_59850650 | caggaaacagctatgacCCCTGCTGAAGAACAGGAAA | 76 |
| chr15_67184470 | tgtaaaacgacggccagtTACTGGGGTGGAGCCTATGA | 77 |
| chr15_67184470 | caggaaacagctatgacTGCCTTTTGGTTCATGTGAC | 78 |
| chr16_74871390 | tgtaaaacgacggccagtTCCTTTGGGATGTTTCCAAC | 79 |
| chr16_74871390 | caggaaacagctatgacCCACTTCAGCCTCCCAAGTA | 80 |
| chr17_38242084 | tgtaaaacgacggccagtCCTCCCAAAGTGCTGTGATT | 81 |
| chr17_38242084 | caggaaacagctatgacATCACCTGAGGTTGCGAGTT | 82 |
| chr18_27921518 | tgtaaaacgacggccagtTGGGAAATACAAAGGCAATG | 83 |
| chr18_27921518 | caggaaacagctatgacTGTTTTGAGGCTTTGGAGAGA | 84 |
| chr18_67786028 | tgtaaaacgacggccagtCTCACTTTATGACGGCAGCA | 85 |
| chr18_67786028 | caggaaacagctatgacGCTTTTTCTGCATCTGTTGG | 86 |
| chr21_32940951 | tgtaaaacgacggccagtAAAGTGCTGGGATGACAGGT | 87 |
| chr21_32940951 | caggaaacagctatgacTGCTGATGTGGGAAACTGAA | 88 |
| chr21_41369734 | tgtaaaacgacggccagtGGGAATTTCTCAATCCACCA | 89 |
| chr21_41369734 | caggaaacagctatgacCTGGCCAGTGGAACCAATAA | 90 |
| chr22_26530011 | tgtaaaacgacggccagtACACCCTCAAGCTTGCTCAC | 91 |
| chr22_26530011 | caggaaacagctatgacATGCACGTGTGAATGGATGT | 92 |
| chr22_47178447 | tgtaaaacgacggccagtTCCTTTCCTCCCTTCCATCT | 93 |
| chr22_47178447 | caggaaacagctatgacTGAAGCTGAGAGACGCAAAA | 94 |
| chrX_47330402 | tgtaaaacgacggccagtTGGCTTGTTAGGAAACCTTCA | 95 |
| chrX_47330402 | caggaaacagctatgacCCATATTAGCCAGGCTGACC | 96 |

PCR primer sequences that were used in amplification are shown in Tables 6 and 7.

TABLE 6
PCR primer sequences for Individual I1C

| | | Reference | | |
|---|---|---|---|---|
| Chromosome | Position | Allele | Variant Allele | Validated |
| chr1 | 14827232 | A | C | Yes |
| chr1 | 21959596 | G | A | Yes |
| chr1 | 62642578 | C | T | Yes |
| chr1 | 158061739 | G | A | Yes |
| chr1 | 176538426 | C | T | Yes |
| chr1 | 197602948 | G | A | Yes |
| chr2 | 32296201 | A | T | Yes |
| chr2 | 58060266 | T | C | Assay failed |
| chr2 | 135596281 | T | C | Yes |
| chr2 | 238760708 | G | T | Yes |
| chr3 | 17614899 | C | T | Yes |
| chr3 | 18023875 | C | T | Yes |
| chr3 | 36828198 | T | C | Yes |
| chr3 | 79639506 | G | A | Assay failed |
| chr3 | 188400668 | G | A | Assay failed |
| chr4 | 28535313 | G | C | Yes |
| chr4 | 32286182 | G | A | Yes |
| chr4 | 38294675 | G | A | Yes |
| chr5 | 5059500 | T | C | Yes |
| chr5 | 19601463 | T | A | Yes |
| chr5 | 133747799 | G | T | Yes |
| chr6 | 63446051 | T | G | Yes |
| chr7 | 77442453 | A | G | Assay failed |
| chr7 | 85735259 | T | C | Yes |
| chr9 | 18393440 | A | G | Yes |
| chr9 | 31764904 | G | C | Assay failed |
| chr9 | 36929059 | A | G | Yes |
| chr9 | 38730375 | G | A | Assay failed |
| chr10 | 92799212 | G | A | Yes |
| chr11 | 18977014 | A | G | Yes |
| chr11 | 74729193 | C | T | Yes |
| chr13 | 43751146 | G | T | Yes |
| chr14 | 27178898 | A | G | Yes |
| chr14 | 38414572 | G | A | Yes |
| chr15 | 59850650 | C | T | Assay failed |
| chr15 | 67184470 | C | T | Yes |
| chr16 | 74871390 | G | C | Yes |
| chr17 | 38242084 | C | G | Assay failed |
| chr18 | 67786028 | C | T | Yes |
| chr21 | 32940951 | A | C | Yes |
| chr21 | 41369734 | C | T | Yes |
| chr22 | 26530011 | C | T | Assay failed |
| chr22 | 47178447 | A | T | Yes |
| chrX | 47330402 | C | T | Yes |

TABLE 7
PCR primer sequences for Individual G1C

| | | Reference | |
|---|---|---|---|
| Chromosome | Position | Allele | Variant Allele |
| chr1 | 49122212 | T | C |
| chr1 | 60146974 | G | T |
| chr1 | 71721920 | C | A |
| chr1 | 1.95E+08 | G | A |
| chr2 | 16980848 | C | T |
| chr2 | 2.02E+08 | C | G |
| chr3 | 30871562 | A | G |
| chr3 | 75185261 | T | A |
| chr3 | 1.04E+08 | A | C |
| chr3 | 1.2E+08 | A | T |
| chr4 | 1.04E+08 | G | A |
| chr4 | 1.78E+08 | A | G |
| chr5 | 98845552 | G | C |
| chr5 | 1.27E+08 | A | G |
| chr6 | 43370795 | C | T |
| chr6 | 1.1E+08 | C | T |
| chr7 | 77496397 | T | C |
| chr7 | 77499437 | T | C |
| chr7 | 83199290 | A | G |
| chr7 | 97182512 | G | A |
| chr8 | 32473276 | C | T |
| chr9 | 26206787 | A | G |
| chr9 | 26217959 | C | G |
| chr9 | 29242240 | C | T |
| chr9 | 87820959 | C | T |
| chr9 | 98020950 | T | C |
| chr10 | 52356602 | T | C |
| chr10 | 1.31E+08 | G | A |
| chr11 | 1.25E+08 | G | A |
| chr12 | 12560227 | C | T |
| chr12 | 22731519 | T | A |
| chr12 | 79850431 | G | A |
| chr12 | 84169467 | T | C |
| chr12 | 90240401 | G | C |
| chr13 | 33181995 | G | T |
| chr13 | 63547484 | C | G |
| chr14 | 52859444 | T | C |
| chr14 | 96580425 | A | G |
| chr15 | 67970619 | G | A |
| chr16 | 81688223 | A | C |
| chr17 | 3046621 | G | A |
| chr17 | 56252548 | C | G |
| chr17 | 72546248 | G | A |
| chr20 | 4419897 | G | A |
| chr20 | 10602218 | C | T |
| chr21 | 43276726 | T | A |

In summary, whole genome sequencing of the offspring (“I1-C”) revealed only 44 high-confidence point mutations (true de novo sites'; Table 5). Taking all positions in the genome at which at least one plasma-derived read had a high-quality mismatch to the reference sequence, and excluding variants present in the parental whole genome sequencing data, 2.5×10⁷ candidate de novo sites were identified, including 39 of the 44 true de novo sites. At baseline, this corresponds to sensitivity of 88.6% with a signal-to-noise ratio of 1-to-6.4×10⁵.

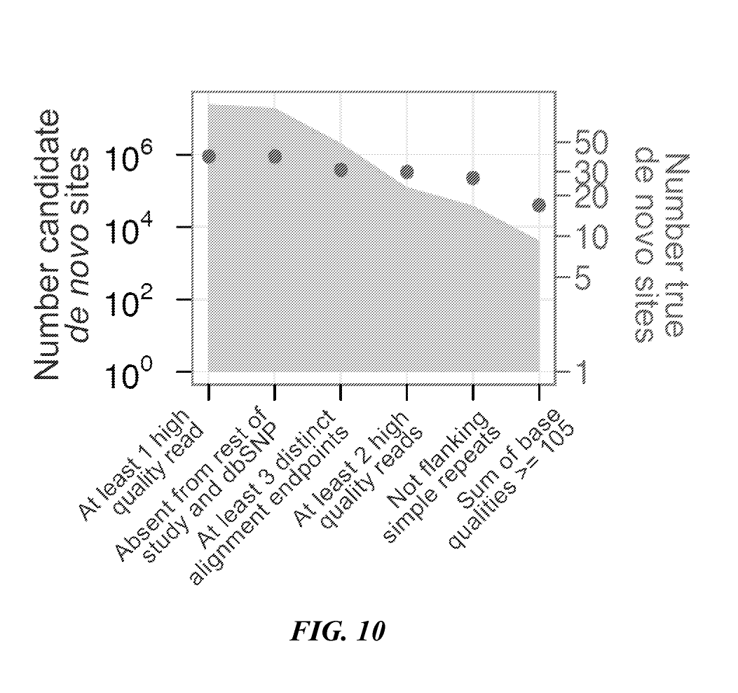

The series of increasingly stringent filters described above and in FIG. 10 were intended to remove sites prone to sequencing or mapping artifacts were applied. Removing alleles also found in at least one read among any other individual sequenced in this study, known polymorphisms from dbSNP (release 135), and sites adjacent to 1-3mer repeats reduced this to 1.8×10⁷ candidate de novo sites. Further requiring at least 2 independent supporting reads, removing sites with excessively many reads supporting the alternate allele (uncorrected P<0.05, per-site one-sided binomial test using fetal-derived fraction of 13%), and requiring supporting base quality scores summing to at least 105 brought the total number of candidate to 3,837, including 17 true de novo sites. This candidate set is substantially depleted for sites of systematic error, and is instead likely dominated by errors originating during PCR, as even a single round of amplification with a proofreading DNA polymerase with an error rate of 1×10⁻⁷ would introduce over 300 candidate sites. This ˜2.800-fold improvement in signal-to-noise ratio reduced the candidate set to a size accessible to validation by targeted methodologies (e.g. an order of magnitude fewer than the number of candidate de novo sites requiring validation in a previous study involving pure genomic DNA from parent-child trios within a nuclear family (Roach et al. 2010)).

### REFERENCES

The references, patents and published patent applications listed below, and all references cited in the specification above are hereby incorporated by reference in their entirety, as if fully set forth herein.
A. Adey et al., Rapid, low-input, low-bias construction of shotgun fragment libraries by high-density in vitro transposition, Genome Biol 11, R119 (2010).
S. S. Ajay, S. C. Parker, H. O. Abaan, K. V. Fajardo, E. H. Margulies, Accurate and comprehensive sequencing of personal genomes. Genome research 21, 1498 (September, 2011).
J. Amberger, C. A. Bocchini, A. F. Scott, A. Hamosh, McKusick's Online Mendelian Inheritance in Man (OMIM). Nucleic acids research 37, D793 (January, 2009).
V. Bansal, V. Bafna, HapCUT: an efficient and accurate algorithm for the haplotype assembly problem, Bioinformatics 24, i153-9 (2008).
Bansal V, et al., An MCMC algorithm for haplotype assembly from whole-genome sequence data. Genome Res. 2008; 18:1336.
C. J. Bell et al., Carrier testing for severe childhood recessive diseases by next-generation sequencing. Science translational medicine 3, 65ra4 (Jan. 12, 2011).
R. W. Chiu et al., Noninvasive prenatal diagnosis of fetal chromosomal aneuploidy by massively parallel genomic sequencing of DNA in maternal plasma. Proceedings of the National Academy of Sciences of the United States of America 105, 20458 (Dec. 23, 2008).
D. F. Conrad et al., Variation in genome-wide mutation rates within and between human families. Nature genetics 43, 712 (July, 2011).
G. M. Cooper, J. Shendure, Needles in stacks of needles: finding disease-causal variants in a wealth of genomic data. Nature reviews. Genetics 12, 628 (September, 2011).
M. A. DePristo et al., A framework for variation discovery and genotyping using next-generation DNA sequencing data, Nature Genetics 43, 491-498 (2011).
Y. M. Dennis Lo, R. W. Chiu, Prenatal diagnosis: progress through plasma nucleic acids. Nature reviews. Genetics 8, 71 (January, 2007).
Jorge Duitama, Thomas Huebsch, Gayle McEwen, Eun-K Suk, ReFHap: a reliable and fast algorithm for single individual haplotyping, Proceedings of the First ACM International Conference on Bioinformatics and Computational Biology (2010) pp 160-169; doi>10.1145/1854776.1854802
R. M. Durbin (corresponding author for The 1000 Genomes Project Consortium) et al., A map of human genome variation from population-scale sequencing. Nature 467, 1061 (Oct. 28, 2010).
H. C. Fan, Y. J. Blumenfeld, U. Chitkara, L. Hudgins, S. R. Quake, Noninvasive diagnosis of fetal aneuploidy by shotgun sequencing DNA from maternal blood. Proceedings of the National Academy of Sciences of the United States of America 105, 16266 (Oct. 21, 2008).
H. C. Fan, J. Wang, A. Potanina, S. R. Quake, Whole-genome molecular haplotyping of single cells. Nature biotechnology 29, 51 (January, 2011).
Dan He, Arthur Choi, Knot Pipatsrisawat, Adnan Darwiche and Eleazar Eskin. Optimal algorithms for haplotype assembly from whole-genome sequence data, Bioinformatics (2010) 26 (12): i183-i190. doi: 10.1093/bioinformatics/btq215
J. O. Kitzman et al., Haplotype-resolved genome sequencing of a Gujarati Indian individual. Nature biotechnology 29, 59 (January, 2011).
Levy S, et al., The diploid genome sequence of an individual human. PLoS Biol. 2007; 5:e254
H. Li, R. Durbin, Fast and accurate short read alignment with Burrows-Wheeler transform, Bioinformatics 25, 1754-1760 (2009).
Y. M. Lo et al., Quantitative analysis of fetal DNA in maternal plasma and serum: implications for noninvasive prenatal diagnosis. American journal of human genetics 62, 768 (April, 1998).
Y. M. Lo et al., Rapid clearance of fetal DNA from maternal plasma. American journal of human genetics 64, 218 (January, 1999).
Y. M. Lo et al., Maternal plasma DNA sequencing reveals the genome-wide genetic and mutational profile of the fetus. Science translational medicine 2, 61 ra91 (Dec. 8, 2010).
L. Ma et al., Direct determination of molecular haplotypes by chromosome microdissection. Nature methods 7, 299 (April, 2010).
A D. G. MacArthur et al., A systematic survey of loss-of-function variants in human protein-coding genes. Science 335, 823 (Feb. 17, 2012).
Hirotaka Matsumoto and Hisanori Kiryu, MixSIH: a mixture model for single individual haplotyping, BMC Genomics 2013, 14(Suppl 2):S5 doi:10.1186/1471-2164-14-S2-S5.
M. A. Nails et al., Imputation of sequence variants for identification of genetic risks for Parkinson's disease: a meta-analysis of genome-wide association studies. Lancet 377, 641 (Feb. 19, 2011).
0. Nygren et al., Quantification of fetal DNA by use of methylation-based DNA discrimination. Clinical chemistry 56, 1627 (October, 2010).
J. C. Roach et al., Analysis of genetic inheritance in a family quartet by whole-genome sequencing. Science 328, 636 (Apr. 30, 2010).
J. Sambrook, Molecular Cloning: A Laboratory Manual, Third Edition (3 volume set) (Cold Spring Harbor Laboratory Press, ed. 3, 2001), p. 2344.
M. W. Snyder et al. Noninvasive fetal genome sequencing: a primer. Prenatal Diagnosis 2013 33, 547-554
P. H. Sudmant et al., Diversity of human copy number variation and multicopy genes, Science 330, 641-646 (2010).
E. K. Suk et al., A comprehensively molecular haplotype-resolved genome of a European individual. Genome research 21, 1672 (October, 2011).
P. D. Stenson et al., The Human Gene Mutation Database: providing a comprehensive central mutation database for molecular diagnostics and personalized genomics. Human genomics 4, 69 (December, 2009).
M. Xie, J. Wang, and T. Jiang, A fast and accurate algorithm for single individual haplotyping, BMC Syst Biol. 2012; 6(Suppl 2); Published online 2012 Dec. 12. doi: 10.1186/1752-0509-6-S2-S8

1. A method of genome sequencing of a fetus comprising:
predicting inheritance or transmission of an allele from one or more maternal-only heterozygous sites from a maternal genomic sequence to a fetal genome sequence; and
predicting inheritance or transmission of an allele from one or more paternal-only heterozygous sites from a paternal genomic sequence to a fetal genome sequence;
wherein the paternal genomic sequence and the maternal genomic sequence are derived from a biological sample containing DNA.

2-4. (canceled)

5. The method of claim 1, wherein the maternal genomic sequence, the paternal genomic sequence, or both, are a haplotype-resolved sequence.

6. The method of claim 1, wherein predicting inheritance or transmission of an allele from one or more maternal-only heterozygous sites comprises:
sequencing a maternal genomic sequence derived from a maternal biological sample;
sequencing a plurality of maternal-fetal cell-free plasma DNA sequences derived from a maternal plasma sample obtained during pregnancy;
determining a percentage of fetal DNA in the maternal plasma sample;
phasing the one or more maternal-only heterozygous sites present in the maternal genomic sequence into one or more haplotype blocks, and
predicting inheritance or transmission of one or more haplotype blocks using a maternal Hidden Markov Model (HMM).

7-13. (canceled)

14. The method of claim 1, wherein predicting inheritance or transmission of an allele from one or more paternal-only heterozygous sites comprises:
sequencing a paternal genomic sequence derived from a paternal biological sample;
sequencing a plurality of maternal-fetal cell-free plasma DNA sequences derived from a maternal plasma DNA sample obtained during pregnancy;
determining a percentage of fetal DNA in the maternal plasma DNA sample;
phasing the one or more paternal-only heterozygous sites present in the maternal genomic sequence into one or more haplotype blocks, and
predicting inheritance or transmission of one or more haplotype blocks using a paternal HMM.

15-20. (canceled)

21. The method of claim 1, further comprising predicting transmission of one or more genomic variants at one or more heterozygous sites that are present on both a maternal genomic sequence and a paternal genomic sequence.

22. The method of claim 1, further comprising predicting one or more de novo mutations in a fetal genomic sequence.

23. The method of claim 22, wherein predicting one or more de novo mutations comprises:
sequencing a paternal genomic sequence from a paternal biological sample;
sequencing a maternal genomic sequence from a maternal biological sample;
sequencing a plurality of maternal-fetal DNA sequences from a maternal plasma sample;
comparing the paternal genomic sequence and the maternal genomic sequence to the maternal-fetal DNA sequences;
identifying one or more candidate de novo alleles as variant alleles observed in the maternal-fetal DNA sequences but not observed in the maternal genomic sequence or the paternal genomic sequence; and
applying a set of filters to the one or more candidate de novo alleles to remove known variants and artifacts from sequencing or mapping, wherein one or more remaining candidate de novo alleles comprise at least one true de novo mutation.

24. The method of claim 23, wherein the set of filters comprise (i) a filter that removes known polymorphisms found in a public database of SNPs; (ii) a filter that removes candidate de novo alleles if the same candidate allele was sequenced with at least moderate base and mapping qualities in another member of the same cohort; (iii) a filter that removes candidate de novo alleles with a flanking “simple repeat” sequence; and (iv) a filter that removes candidate de novo alleles with an excess reads supporting the de novo mutation relative to the expectation based on the estimated fetal proportion.

25. (canceled)

26. The method of claim 23, wherein the maternal genomic sequence, the paternal genomic sequence, and the fetal genomic sequence are sequenced by shotgun sequencing, clone pool sequencing, or a combination of both.

27. A method for predicting inheritance of a maternal-only genetic abnormality comprising:
sequencing a maternal genomic sequence derived from a maternal biological sample;
sequencing a plurality of maternal-fetal cell-free plasma DNA sequences derived from a maternal plasma sample obtained during pregnancy;
determining a percentage of fetal DNA in the maternal plasma sample;
phasing the one or more maternal-only heterozygous sites present in the maternal genomic sequence into one or more haplotype blocks, and
predicting inheritance or transmission of one or more haplotype blocks using a maternal Hidden Markov Model (HMM), the maternal HMM comprising
a set of latent inheritance states at each maternal heterozygous site;
a probability model for transitions between latent states; and
an emission probability model for each latent state.

28. The method of claim 27, further comprising inferring transitions within the one or more haplotype blocks using the maternal HMM.

29. (canceled)

30. The method of claim 27, further comprising:
calculating a probability of observing a first maternal-inherited allele that is the same as a paternal-inherited homozygous allele from a maternal heterozygous site using the emission probability model; and
calculating a probability of observing a second maternal-inherited allele that differs from a paternal-inherited homozygous allele from a maternal heterozygous site using the emission probability model.

31. The method of claim 30, wherein tithe emission probability of observing the sequencing data supporting the first maternal-inherited allele is calculated using Equation 1, (ii) the emission probability of observing the sequencing data supporting the second maternal-inherited allele is calculated using Equation 2, or (iii) both (i) and (ii).

32. (canceled)

33. The method of claim 28, wherein an inferred transition using the maternal HMM represents a true recombination event or a switch error in phasing.

34. The method of claim 27, further comprising predicting inheritance of an allele from one or more maternal-only heterozygous sites using a site-by-site analysis.

35. A method for predicting inheritance of a paternal-only genetic abnormality comprising:
sequencing a paternal genomic sequence derived from a paternal biological sample;
sequencing a plurality of maternal-fetal cell-free plasma DNA sequences derived from a maternal plasma DNA sample obtained during pregnancy;
determining a percentage of fetal DNA in the maternal plasma DNA sample;
phasing the one or more paternal-only heterozygous sites present in the maternal genomic sequence into one or more haplotype blocks, and
predicting inheritance or transmission of one or more haplotype blocks using a paternal HMM, the paternal HMM comprising
a set of latent inheritance states at each maternal heterozygous site;
a probability model for transitions between latent states; and
an emission probability model for each latent state.

36. The method of claim 35, further comprising inferring transitions within the one or more haplotype blocks using the paternal Hidden Markov Model (HMM).

37. (canceled)

38. The method of claim 35, wherein the paternal HMM comprises
calculating a probability of observing a first paternal-inherited allele that is the same as a maternal-inherited homozygous allele from a paternal heterozygous site using the emission probability model; and
calculating a probability of observing a second paternal-inherited allele that differs from a maternal-inherited homozygous allele from a paternal heterozygous site using the emission probability model.

39. The method of claim 38, wherein (i) the emission probability of observing the sequencing data supporting the first paternal-inherited allele is calculated using Equation 3, (ii) the emission probability of observing the sequencing data supporting the second paternal-inherited allele is calculated using Equation 4, or (iii) both (i) and (ii).

40. (canceled)

41. The method of claim 36, wherein an inferred transition using the paternal HMM represents a true recombination event or a switch error in phasing.

42-45. (canceled)
